(19) United States
(12) Patent Application Publication
(10) Pub. No.: US 2008/0194596 A1
(43) Pub. Date: Aug. 14, 2008

(54) Therapeutic Combination Including a Selective Erbb2 Inhibitor

(75) Inventor: Stephen Paul Letrent, San Diego, CA (US)

Correspondence Address: PFIZER INC, 10555 SCIENCE CENTER DRIVE, SAN DIEGO, CA, 92121, US

(73) Assignee: FRIZER INC.

(21) Appl. No.: 11/914,924

(22) PCT Filed: May 22, 2006
(86) PCT No.: PCT/IB2006/001402

§ 371 Date: Nov. 19, 2007

Related U.S. Application Data
(60) Provisional application No. 60/687,087, filed on Jun. 3, 2005.

(51) Int. Cl. A61K 31/517; A61P 35/00

(52) U.S. Cl. 514/266.4

(57) ABSTRACT

This invention relates to a method of treatment of cancer with a combination of an additional therapeutic agent in mammals. This invention also relates to a kit useful in the treatment of abnormal cell growth in mammals, especially humans.

### CROSS-REFERENCE TO RELATED APPLICATION(S)

Reference is made to U.S. Provisional Application Ser. No. 60/517,636, filed Nov. 6, 2003. Reference is also made to U.S. Provisional Application Ser. No. 60/549,600, filed Mar. 3, 2004. Reference is also made to U.S. Pat. No. 6,890,924, filed Jun. 18, 2001. The disclosures of each of these applications are incorporated herein by reference.

### BACKGROUND OF THE INVENTION

This invention relates to a method of cancer treatment with a combination of an erbB2 inhibitor and an antibody, in mammals. More particularly, this invention relates to a method of treating cancer by administering an erbB2 ligand in combination with an erbB antibody. This invention also relates to a kit useful in the treatment of abnormal cell growth in mammals, especially humans.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor (EGF), a transmembrane domain, and an intracellular portion which functions as a kinase to phosphorylate specific tyrosine residues in proteins and hence to influence cell proliferation. The EGF receptor tyrosine kinase family has four members: EGFR (HERI, erbB1); HER2 (c-erbB2, erbB2, neu); HER3 (erbB3); and HER4 (erbB4). The ErbB receptors generally transduce signals through two pathways. It is known that such kinases are frequently and aberrantly expressed in common human cancers such as breast cancer, gastrointestinal cancer of colon, rectum or stomach, leukemia, and ovarian, bronchial or pancreatic cancer. It has also been shown that epidermal growth factor receptor (EGFR), which possesses tyrosine kinase activity, is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma which expresses epidermal growth factor receptor tyrosine kinase (EGFR) but is without effect on the growth of another carcinoma which does not express the EGF receptor.

Certain compounds useful in the treatment of cancer are disclosed in WO 01/98277, the disclosure of which is incorporated herein in its entirety. Various other compounds, such as styrene derivatives, have also been shown to possess tyrosine kinase inhibitory properties. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO096/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are U.S. patent application Ser. No. 09/488,350 (filed Jan. 20, 2000) and Ser. No. 09/488,378 (filed Jan. 20, 2000), both of which are incorporated herein by reference in their entirety.

Antibodies to erbB2 are known and have therapeutic utility. U.S. Pat. No. 5,725,856 is directed, in part, to treatment by administering an antibody that binds to the extracellular domain of the erbB2 (HER2) receptor. U.S. Pat. No. 5,677,171 is directed to a monoclonal antibody that binds the HER2 receptor. U.S. Pat. No. 5,720,954 is directed to a treatment by use of a cytotoxic factor and an antibody to HER2 receptor. U.S. Pat. No. 5,770,195 is directed to inhibiting the growth of tumor cells. U.S. Pat. No. 6,165,464 is directed to an isolated human antibody that binds HER2 receptor. U.S. Pat. No. 6,387,371 is directed to a method of treating a cancer by administering an antibody and a factor which suppresses cancer cell growth.

### SUMMARY OF THE INVENTION

In one aspect the present invention comprises a method of treating a mammal having abnormal cell growth, such as cancer, comprising: administering to said mammal in need of such treatment, sequentially in either order, simultaneously, or both, (i) a therapeutically effective amount of a compound of the formula 1

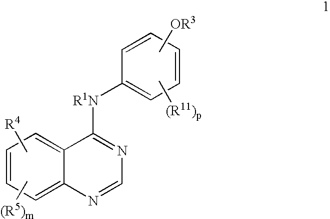

and to pharmaceutically acceptable salts, solvates and prodrugs thereof, wherein:

m is an integer from 0 to 3;

p is an integer from 0 to 4;

each R¹ and R² is independently selected from H and C₁-C₆ alkyl;

R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;

R⁴ is —(CR¹⁶R¹⁷)ₘ—C≡C—(CR¹⁶R¹⁷)ₜR⁹, —(CR¹⁶R¹⁷)ₘ—C═C—(CR¹⁶R¹⁷)ₜ—R⁹, —(CR¹⁶R¹⁷)ₘ—C≡C—(CR¹⁶R¹⁷)ₖR¹³, —(CR¹⁶R¹⁷)ₘ—C═C—(CR¹⁶R¹⁷)ₖR¹³, or —(CR¹⁶R¹⁷)ₜR⁹, wherein the attachment point to R⁹ is through a carbon atom of the R⁹ group, each k is an integer from 1 to 3, each t is an integer from 0 to 5, and each m is an integer from 0 to 3;

each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;

each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R² trifluoromethyl, trifluoromethoxy, C₁-C₆ alkyl, C₂-C₆ alkenyl, C₂-C₆ alkynyl, hydroxy, and C₁-C₆ alkoxy;

or R⁶ and R⁷, or R^{6a} and R⁷, when attached to a nitrogen atom (including the same nitrogen atom or two separate nitrogen atoms in proximity to each other through interconnection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;

each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;

R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;

each R¹¹ is independently selected from the substituents provided in the definition of R⁸ except R¹¹ is not oxo(═O);

R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;

R¹³ is —NR¹R¹⁴ or —OR¹⁴;

R¹⁴ is H, R¹⁵, —C(O)R¹⁵, —SO₂R¹⁵, —C(O)NR¹⁵R⁷, —SO₂NR¹⁵R⁷, or —CO₂R¹⁵;

R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;

each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;

R¹⁸ is C₁-C₆ alkyl wherein each carbon not bound to a N or O atom, or to S(O)ⱼ, wherein j is an integer from 0 to 2, is optionally substituted with R¹²;

and wherein any of the above-mentioned substituents comprising a CH₃ (methyl), CH₂ (methylene), or CH (methine) group, which is not attached to a halogeno, SO or SO₂ group or to a N, O or S atom, is optionally substituted with a group selected from hydroxy, halo, C₁-C₄ alkyl, C₁-C₄ alkoxy and —NR¹R²;

and (ii) a therapeutically effective amount of an additional therapeutic agent.

In a specific embodiment of the present invention, R³ is —(CR¹R²)ₜ(4 to 10 membered heterocyclic), wherein t is an integer from 0 to 5; said heterocyclic group is optionally fused to a benzene ring or a C₅-C₈ cycloalkyl group, and the foregoing R³ groups, including any optional fused rings referred to above, are optionally substituted by 1 to 3 R⁸ groups.

Other specific embodiments of the compounds of formula 1 include those wherein R³ is —(CR¹R²)ₜ(4 to 10 membered heterocyclic), wherein t is an integer from 0 to 5, and the foregoing R³ groups are optionally substituted by 1 to 3 R⁸ groups.

Other specific embodiments of the compounds of formula 1 include those wherein R³ is selected from

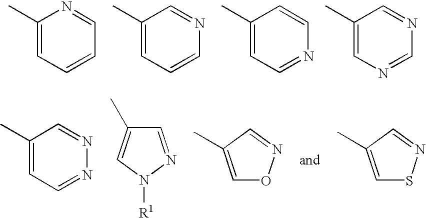

wherein the foregoing R³ groups are optionally substituted by 1 to 3 R⁸ groups.

Other specific embodiments of the compounds of formula 1 include those wherein R³ is pyridin-3-yl optionally substituted by 1 to 3 R⁸ groups.

Other specific embodiments of the compounds for formula 1 include those wherein R⁴ is —(CR¹⁶R¹⁷)ₘ—C≡C—(CR¹⁶R¹⁷)ₜR⁹, wherein m is an integer from 0 to 3, and t is an integer from 0 to 5.

Other specific embodiments of the compounds for formula 1 include those wherein R⁴ is —(CR¹⁶R¹⁷)ₘ—C≡C—(CR¹⁶R¹⁷)ₜR⁹, wherein m is an integer from 0 to 3, and t is an integer from 0 to 5, wherein R⁹ is selected from 3-piperidinyl and 4-piperidinyl each of which is optionally substituted with 1 or 2 R⁸ groups.

Other specific embodiments of the compounds for formula 1 include those wherein R⁴ is —(CR¹⁶R¹⁷)ₘ—C═C—(CR¹⁶R¹⁷)ₜ—R⁹, wherein m is an integer from 0 to 3, and t is an integer from 0 to 5.

Other specific embodiments of the compounds for formula 1 include those wherein R⁴ is —(CR¹⁶R¹⁷)ₘ—C═C—(CR¹⁶R¹⁷)ₜ—R⁹, wherein m is an integer from 0 to 3, and t is an integer from 0 to 5, wherein R⁹ is selected from 3-piperidinyl and 4-piperidinyl (optionally substituted with 1 or 2 R⁸ groups).

Other specific embodiments of the compounds for formula 1 include those wherein R⁴ is —(CR¹⁶R¹⁷)ₘ—C≡C—(CR¹⁶R¹⁷)ₖR¹³, wherein k is an integer from 1 to 3 and m is an integer from 0 to 3.

Other specific embodiments of the compounds for formula 1 include those wherein R⁴ is —(CR¹⁶R¹⁷)ₘ—C≡C—(CR¹⁶R¹⁷)ₖR¹³, wherein k is an integer from 1 to 3 and m is an integer from 0 to 3, wherein R is —NR¹R¹⁴, wherein R¹⁴ is selected from —C(O)R¹⁵, —SO₂R¹⁵, and C(O)NR¹⁵R⁷.

Other specific embodiments of the compounds for formula 1 include those wherein R⁴ is —(CR¹⁶R¹⁷)ₘ—C═C—(CR¹⁶R¹⁷)ₖR¹³, wherein k is an integer from 1 to 3 and m is an integer from 0 to 3.

Other specific embodiments of the compounds for formula 1 include those wherein R⁴ is —(CR¹⁶R¹⁷)ₘ—C═C—(CR¹⁶R¹⁷)ₖR¹³, wherein k is an integer from 1 to 3 and m is an integer from 0 to 3, wherein R is —NR¹R¹⁴, wherein R¹⁴ is selected from —C(O)R¹⁵, —SO₂R¹⁵, and —C(O)NR¹⁵R⁷.

Other specific embodiments of the compounds for formula 1 include those wherein R⁴ is —(CR¹⁶R¹⁷)ₘ—C≡C—(CR¹⁶R¹⁷)ₖR¹³ or —(CR¹⁶R¹⁷)ₘ—C═C(CR¹⁶R¹⁷)ₖR¹³, wherein k is an integer from 1 to 3 and m is an integer from 0 to 3, R¹³ is —NR¹R¹⁴ or —OR¹⁴, R¹⁴ is R¹⁵, R¹⁵ is R¹⁸, and R¹⁸ is C₁-C₆ alkyl optionally substituted by —OR⁶, —S(O)ⱼR⁶, —NR⁶R⁷, —NR⁶C(O)R⁷, —NR⁶SO₂R⁷, —NR⁶CO₂R⁷, CN, —C(O)R⁶, or halo.

In yet another aspect, the method of the invention comprises treatment of a cancer that overexpresses an erbB2 protein. In a particular embodiment, the level of expression of erbB2 is +2 or +3 on a four-value scale that ranges from 0 (normal) to +1 to +2 to +3. A value of +3 is associated with highly aggressive tumors.

Specific preferred compounds of the methods and kits of the present invention include those including one or more of the following compounds:

(±)-[3-Methyl-4-(pyridin-3-yloxy)-phenyl]-(6-piperidin-3-ylethynyl-quinazolin-4-yl)-amine;

2-Methoxy-N-(3-{4-[3-methyl-4-(pyridin-3-yloxy)-phenylamino]-quinazolin-6-yl}-prop-2-ynyl)-acetamide

(±)-[3-Methyl-4-(6-methyl-pyridin-3-yloxy)-phenyl]-(6-piperidin-3-ylethynyl-quinazolin-4-yl)-amine;

[3-Methyl-4-(6-methyl-pyridin-3-yloxy)-phenyl]-(6-piperidin-4-ylethynyl-quinazolin-4-yl)-amine;

2-Methoxy-N-(3-{4-[3-methyl-4-(6-methyl-pyridin-3-yloxy)-phenylamino]-quinazolin-6-yl}-prop-2-ynyl)-acetamide;

2-Fluoro-N-(3-{4-[3-methyl-4-(6-methyl-pyridin-3-yloxy)-phenylamino]-quinazolin-6-yl}-prop-2-ynyl)-acetamide;

E-2-Methoxy-N-(3-{4-[3-methyl-4-(6-methyl-pyridin-3-yloxy)-phenylamino]-quinazolin-6-yl}-allyl)-acetamide;

[3-Methyl-4-(pyridin-3-yloxy)-phenyl]-(6-piperidin-4-ylethynyl-quinazolin-4-yl)-amine;

2-Methoxy-N-(1-{4-[3-methyl-4-(6-methyl-pyridin-3-yloxy)-phenylamino]-quinazolin-6-ylethynyl}-cyclopropyl)-acetamide;

E-N-(3-{4-[3-Chloro-4-(6-methyl-pyridin-3-yloxy)-phenylamino]-quinazolin-6-yl}-allyl)-2-methoxy-acetamide;

N-(3-{4-[3-Chloro-4-(6-methyl-pyridin-3-yloxy)-phenylamino]-quinazolin-6-yl}-prop-2-ynyl)-acetamide;

N-(3-{4-[3-Methyl-4-(6-methyl-pyridin-3-yloxy)-phenylamino]-quinazolin-6-yl}-prop-2-ynyl)-acetamide;

E-N-(3-{4-[3-Chloro-4-(6-methyl-pyridin-3-yloxy)-phenylamino]-quinazolin-6-yl}-allyl)-acetamide;

E-2-Ethoxy-N-(3-{4-[3-methyl-4-(6-methyl-pyridin-3-yloxy)-phenylamino]-quinazolin-6-yl}-allyl)-acetamide;

1-Ethyl-3-(3-{4-[3-methyl-4-(6-methyl-pyridin-3-yloxy)-phenylamino]-quinazolin-6-yl}-prop-2-ynyl)-urea;

Piperazine-1-carboxylic acid (3-{4-[3-methyl-4-(6-methyl-pyridin-3-yloxy)-phenylamino]-quinazolin-6-yl}-prop-2-ynyl)-amide;

(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;

2-Dimethylamino-N-(3-{4-[3-methyl-4-(pyridin-3-yloxy)-phenylamino]-quinazolin-6-yl}-prop-2-ynyl)-acetamide;

E-N-(3-{4-[3-Methyl-4-(6-methyl-pyridin-3-yloxy)-phenylamino]-quinazolin-6-yl}-allyl)-methanesulfonamide;

Isoxazole-5-carboxylic acid (3-{4-[3-methyl-4-(6-methyl-pyridin-3-yloxy)-phenylamino]-quinazolin-6-yl}-prop-2-ynyl)-amide;

1-(1,1-Dimethyl-3-{4-[3-methyl-4-(6-methyl-pyridin-3-yloxy)-phenylamino]-quinazolin-6-yl}-prop-2-ynyl)-3-ethyl-urea;

and the pharmaceutically acceptable salts, prodrugs and solvates of the foregoing compounds.

The present invention also provides a combination of the present invention, i.e., a combination of a compound of formula I and one or more additional therapeutic agents selected from the group consisting of an antitumor agent, alkylating agent, antimetabolite, antibiotic, plant-derived antitumor agent, camptothecin derivative, tyrosine kinase inhibitor, antibody, interferon, and biological response modifier.

In one embodiment, The additional therapeutic agent is selected from the group consisting of 5-fluoruracil, bevacizumab, bicalutamide, buserelin, carboplatin, cetuximab, cisplatin, CP-547,632, CP-751,871, ticilimumab (CP-675,206), cyproterone acetate, DES (diethylstilbestrol), dexamethasone, docetaxel, doxorubicin, epirubicin, erlotinib, estramustine, exemestane, flutamide, gefitinib, gemcitabine, goserelin, hydrocortisone, irinotecan (oral or IV), ixabepilone (BMS-247550) ketoconazole, letrozole, leuprolide, mitoxantrone, nilutamine, oxaliplatin, paclitaxel, pertuzumab, prednisone, PROMUNE™ (CP-7909, PF-3512676), SU-11248, tamoxifen, and vinorelbine.

In a particular embodiment, the invention provides a combination comprising a compound of formula I and one or more agents selected from paclitaxel, exemestane, tamoxifen, and combinations thereof.

The method of the invention also relates to a method for the treatment of abnormal cell growth in a mammal, including a human, comprising administering to said mammal an amount of a compound of the formula 1, as defined above, or a pharmaceutically acceptable salt, solvate or prodrug thereof, that is effective in treating abnormal cell growth in combination with an additional therapeutic agent. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In another aspect the method of the invention is directed to the combination of step (i), above, and step (ii) above, in which the combination is synergistic compared to either alone. Preferably, the combination is superadditive.

This invention also relates to a kit for treatment of abnormal cell growth, comprising an agent of formula 1 as defined above, and written instructions for simultaneous administration with at least one additional therapeutic agent. In a particular aspect the written instructions specify the administration of E-2-Methoxy-N-(3-{4-(6-methyl-pyridin-3-yloxy)-phenylamino]-quinazolin-6-yl}-allyl)-acetamide. In one embodiment of said kit, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said kit, said abnormal cell growth is a benign proliferative disease, including, but not limited to, psoriasis, benign prostatic hypertrophy or restinosis.

The compounds of formula 1, and the pharmaceutically acceptable salts, solvates and prodrugs thereof, can also be used in combination with signal transduction inhibitors, such as agents that can inhibit EGFR (epidermal growth factor receptor) responses, such as EGFR antibodies, EGF antibodies, and molecules that are EGFR inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include Herceptin, 2C4, and pertuzumab. Such inhibitors also include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “halo”, as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl”, as used herein, unless otherwise indicated, includes an organic radical derived from an aromatic hydrocarbon by removal of one hydrogen, such as phenyl or naphthyl.

The term “alkoxy”, as used herein, unless otherwise indicated, includes —O-alkyl groups wherein alkyl is as defined above.

The term “4 to 10 membered heterocyclic”, as used herein, unless otherwise indicated, includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The term “Me” means methyl, “Et” means ethyl, and “Ac” means acetyl.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups that have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

The subject matter of the invention also includes isotopically-labelled compounds, and the pharmaceutically acceptable salts, solvates and prodrugs thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C ¹⁴C, ¹⁵N, ¹⁸O, ¹⁷O, ³⁵S, ¹⁸F, and ³⁵Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The terms synergy and synergistic mean that the combination of two or more effectors or active agents is at least additive in their effect. Preferably, the synergy is greater than additive. More preferably, the synergy is superadditive. The term “additive” is use to mean that the result of the combination of the two or more effectors or agents is more than the sum of each effector or agent together and preferably at least 10 percent greater than the combination's additive effect. The term “superadditive” is used to mean that the result of combination of two or more effectors is at least 25 percent greater than the combination's additive effect.

“Ligand” is particularly used to describe a small molecule that binds to a receptor. An important class of ligands in the instant invention are those of formula 1 which bind to receptors in the epidermal growth factor family. Ligands can be inhibitors of receptor function and can be antagonists of the action of activators.

Certain abbreviations common in the art are freely used and will be understood in context. Among these are pharmacokinetics (PK), pharmacodynamics (PD), fetal bovine serum (FBS), penicillin/streptomycin (pen/strep), Roswell Park Memorial Institute (RPMI), per os (PO), once per day (QD), interaperitoneally (IP), subcutaneously (SC), enzyme-linked immunosorbent assay (ELISA), the maximum concentration of an analyte in a PK analysis (Cₘₐₓ), and the average concentration of an analyte in a PK analysis (Cₐᵥₑ).

### DETAILED DESCRIPTION OF THE INVENTION

The compounds of formula 1 may be prepared according to the synthetic scheme outlined in Scheme I below.

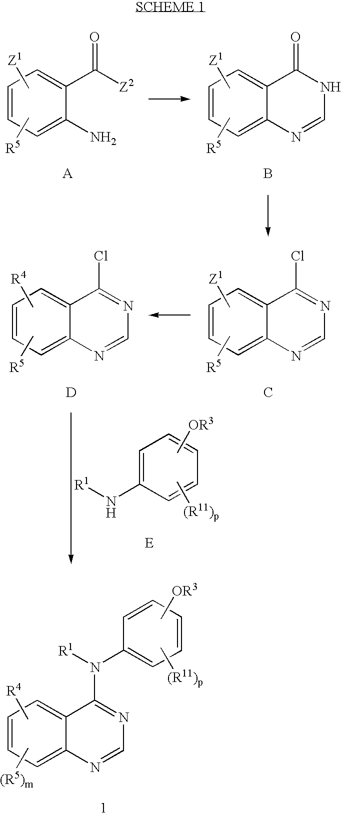

General synthetic methods which may be referred to for preparing the compounds of the present invention are provided in U.S. Pat. No. 5,747,498 (issued May 5, 1998), U.S. patent application Ser. No. 08/953078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in U.S. patent application Ser. No. 09/488,350 (filed Jan. 20, 2000) and Ser. No. 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem. 1986, 51, 5, p. 616. Palladium-catalyzed boronic acid couplings are described in Miyaura, N., Yanagi, T., Suzuki, A. Syn. Comm. 1981, 11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

Alternatively terminal alkynes may be prepared by a twostep procedure. First, the addition of the lithium anion of TMS (trimethylsilyl) acetylene to an appropriately substituted/protected aldehyde as in: Nakatani, K. et. al. Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al. Tet. Lett., 31, 1, 1990, 59.

Starting materials, the synthesis of which is not specifically described above, are either commercially available or can be prepared using methods well known to those of skill in the art.

In each of the reactions discussed or illustrated in the Schemes above, pressure is not critical unless otherwise indicated. Pressures from about 0.5 atmospheres to about 5 atmospheres are generally acceptable, and ambient pressure, i.e., about 1 atmosphere, is preferred as a matter of convenience.

With reference to Scheme 1 above, the compound of formula 1 may be prepared by coupling the compound of formula D wherein R⁴ and R⁵ are defined above, with an amine of formula E wherein R¹, R³ and R¹¹ are as defined above, in an anhydrous solvent, in particular a solvent selected from DMF (N,N-dimethylformamide), DME (ethylene glycol dimethyl ether), DCE (dichloroethane) and t-butanol, and phenol, or a mixture of the foregoing solvents, a temperature within the range of about 50-150° C. for a period ranging from 1 hour to 48 hours. The heteroaryloxyanilines of formula E may be prepared by methods known to those skilled in the art, such as, reduction of the corresponding nitro intermediates. Reduction of aromatic nitro groups may be performed by methods outlined in Brown, R. K., Nelson, N. A. J. Org. Chem. 1954, p. 5149; Yuste, R., Saldana, M, Walls, F., Tet. Lett. 1982, 23, 2, p. 147; or in WO 96/09294, referred to above. Appropriate heteroaryloxy nitrobenzene derivatives may be prepared from halo nitrobenzene precursors by nucleophilic displacement of the halide with an appropriate alcohol as described in Dinsmore, C. J. et. al., Bioorg. Med. Chem. Lett., 7, 10, 1997, 1345; Loupy, A. et. al., Synth. Commun., 20, 18, 1990, 2855; or Brunelle, D. J., Tet. Lett., 25, 32, 1984, 3383. Compounds of formula E in which R¹ is a C₁-C₆ alkyl group may be prepared by reductive amination of the parent aniline with R¹CH(O). The compound of formula D may be prepared by treating a compound of formula C, wherein Z¹ is an activating group, such as bromo, iodo, —N₂, or —OTf (which is —OSO₂CF₃), or the precursor of an activating group such as NO₂, NH₂ or OH, with a coupling partner, such as a terminal alkyne, terminal alkene, vinyl halide, vinyl stannane, vinylborane, alkyl borane, or an alkyl or alkenyl zinc reagent. The compound of formula C can be prepared by treating a compound of formula B with a chlorinating reagent such as POCl₃, SOCl₂ or ClC(O)C(O)Cl/DMF in a halogenated solvent at a temperature ranging from about 60° C. to 150° C. for a period ranging from about 2 to 24 hours. Compounds of formula B may be prepared from a compound of formula A wherein Z¹ is as described above and Z² is NH₂, C₁-C₆ alkoxy or OH, according to one or more procedures described in WO 95/19774, referred to above.

Any compound of formula 1 can be converted into another compound of formula 1 by standard manipulations to the R⁴ group. These methods are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively; c) treatment of phenyl (or substituted phenyl) carbamates with primary of secondary amines to form the corresponding ureas as in Thavonekham, B et. al. Synthesis (1997), 10, p 1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p 1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formulas 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product. Since a single compound of the present invention may include more than one acidic or basic moieties, the compounds of the present invention may include mono, di or tri-salts in a single compound.

The method of the invention comprises treating a mammal having a cancer, comprising: administering to said mammal in need of such treatment, sequentially in either order, simultaneously, or both, (i) a therapeutically effective amount of a compound of the formula 1, as defined above, and (ii) a therapeutically effective amount of an additional therapeutic agent. In a preferred embodiment, the method of the invention comprises treating a mammal having a cancer, comprising: administering to said mammal in need of such treatment, sequentially in either order, simultaneously, or both, (i) a therapeutically effective amount of a compound of the formula 1, as defined above, and (ii) a therapeutically effective amount of an additional therapeutic agent.

The cancer can be a solid cancer. In a particular aspect the cancer is not a solid tumor, including, for example, a leukemia or a lymphoma. The volume of the solid cancer can decrease upon administration of the method of the invention.

The amount of additional therapeutic agents may be administered as doses in the range of 1-20 mg/kg anywhere from once or twice daily to once every 3 to 4 weeks. The amount of the additional therapeutic agent administered may be less than about 2 mg/kg/week. In one aspect, the amount of the additional therapeutic agent administered may be about 0.6 mg/kg/week. The additional agent can be administered at least about once per week. In another aspect, the additional agent can be administered about once per two weeks.

For example, paclitaxel may be administered in a dosage of 5 to 500 mg/m² via IV infusion over 0.5 to 24 hours or orally once every 1 to 4 weeks or daily for 5 days repeated every 1 to 4 weeks, for example, 15-420 mg/m² dose over a 6 to 24 hour infusion.

Docetaxel may be administered in a dosage of 50 to 200 mg/m² via IV infusion over 0.5 to 24 hours once every 1 to 4 weeks or daily for 5 days repeated every 1 to 4 weeks. For example, docetaxel may be administered in a dosage of 60-100 mg/m² IV over 1 hour, every three weeks.

Carboplatin may be administered in a dosage of 100 to 500 mg/m² via IV infusion over 0.5 to 24 hours once every 1 to 4 weeks or daily for 5 days repeated every 1 to 4 weeks, for example, 360 mg/m² IV infusion on day 1 every 4 weeks. Alternatively, the dosage may be varied in order to achieve a target area under the curve of 2 to 10 as derived by use of creatinine clearance (estimate or actual) known to those so skilled in the art.

Cisplatin may be administered in a dosage of 50 to 200 mg/m² via IV infusion over 0.5 to 24 hours once every 1 to 4 weeks or daily for 5 days repeated every 1 to 4 weeks, for example, 75-100 mg/m² via IV infusion once every 4 weeks.

CP-751,871, which is currently under development by Pfizer Inc., may be administered in a dosage of 0.1 to 10 mg/kg via IV infusion over 0.5 to 24 hours once every 1 to 8 weeks.

CP-675,206 (ticilimumab) which is currently under development by Pfizer Inc., may be administered in a dosage of 0.1 to 10 mg/kg via IV infusion over 0.5 to 24 hours once every 1 to 8 weeks.

Oxaliplatin may be administered in a dosage of 50 to 100 mg/m² via IV infusion over 0.5 to 24 hours once every 1 to 4 weeks or daily for 5 days repeated every 1 to 4 weeks, for example, 85 mg/m² IV infusion over 120 minutes once every 2 weeks.

Irinotecan may be administered in a dosage of 100 to 300 mg/m² via IV infusion over 0.5 to 24 hours once every 1 to 4 weeks or daily for 5 days repeated every 1 to 4 weeks, for example, 125 mg/m² or 180 mg/m² via IV infusion over 90 minutes per week for 4 weeks.

Ixabepilone (BMS-247550), which is currently under development by Bristol-Myers Squibb, may be administered in a dosage of 5 to 65 mg/m² via IV infusion over 0.5 to 24 hours once every 1 to 4 weeks or daily for 5 days repeated every 1 to 4 weeks.

5-fluorouracil may be administered in a dosage of 1 to 1000 mg/m² or mg/kg via IV infusion over 0.5 to 24 hours once every 1 to 4 weeks or daily for 2 to 7 days repeated every 1 to 4 weeks. For example, 5-fluorouracil may be administered in a dosage of 12 mg/kg via IV infusion once daily for 4 successive days.

Vinorelbine may be administered in a dosage of 10 to 100 mg/m² via IV infusion over 1 to 120 minutes once every 1 to 4 weeks, for example, 25 or 30 mg/m² over 6 to 10 minutes administered weekly.

Vinflunine, which is currently under development by Bristol-Myers Squibb and Pierre Fabre Médicament, may be administered in a dosage of 100 to 500 mg/m² administered by IV infusion over 0.5 to 24 hours once every 1 to 4 weeks or daily for 4 to 8 days repeated every 1 to 4 weeks.

Gemcitabine may be administered in a dosage of 500 to 1000 mg/m² via IV infusion over 0.5 to 24 hours once every 1 to 7 weeks, for example 1000 mg/m² IV infusion over 30 minutes once weekly for up to 7 weeks.

Doxorubicin may be administered in a dosage of 10 to 100 mg/m² via IV infusion over 0.5 to 24 hours once every 1 to 4 weeks, for example, 40 to 75 mg/m² once every 21-28 days.

Epirubicin may be administered in a dosage of 10 to 200 mg/m² via IV infusion over 0.5 to 24 hours once every 1 to 4 weeks, for example, 100 to 120 mg/m² once every 3 to 4 weeks.

Bevacizumab may be administered in a dosage of 1 to 20 mg/kg via IV infusion over 0.5 to 24 hours once very 1 to 8 weeks with or without a loading dose of 1 to 20 mg/kg, for example, 5 mg/kg given once every 2 weeks.

Gefitinib may be administered orally in a dosage of 250 to 1000 mg 1 to 2 times per day, for example, a 250 mg tablet.

Erlotinib may be administered orally in a dosage of 150 to 300 mg 1 to 2 times per day, for example, a daily dose is 150 mg.

Cetuximab may be administered in a dosage of 100 to 600 mg/m² via IV infusion over 0.5 to 24 hours once very 1 to 8 weeks with or without a loading dose of 100 to 1000 mg/m^{2,} for example, 400 mg/m² as an initial loading dose administered as a 120-minute IV (maximum infusion rate 5 mL/min) and a weekly dose of 250 mg/m² via IV infusion over 60 minutes (maximum infusion rate 5 mL/min).

Pertuzumab, which is currently under development by Genentech, may be administered in a dosage of 100 to 2000 mg via IV infusion over 0.5 to 24 hours once every 1 to 8 weeks with or without a loading dose of 100 to 2000 mg.

Exemestane may be administered orally in a dosage of 25 to 50 mg once a day, for example, a daily dose of 25 mg.

Letrozole may be administered orally in a dosage of 2.5 to 5 mg administered 1 to 2 times per day, for example a daily dose of 2.5 mg.

Tamoxifen may be administered orally in a dosage of 20 to 100 mg 1 to 2 times per day, for example, a daily dose of 20-40 mg.

Bicalutamide may be administered orally in a dosage of 50 to 200 mg 1 to 2 times per day, for example, a daily dose of 50 mg.

Flutamide may be administered orally in a dosage of 125 to 500 mg 1 to 4 times per day, for example, 250 mg 3 times a day at 8-hour intervals for a total dose of 750 mg.

Nilutamide may be administered orally in a dosage of 100 to 600 mg 1 to 2 times per day, for example, 300 mg once a day.

Dexamethasone may be administered orally in a dosage of 0.5 to 10 mg 1 to 4 times per day.

Ketoconazole may be administered orally in a dosage of 100 to 400 mg 1 to 4 times per day.

SU-11248, which is currently under development by Pfizer Inc., may be administered orally in a dosage of 1 to 100 mg 1 to 2 times every day or daily for 1 to 4 weeks followed by a 1 to 4 week break repeated.

Goserelin may be administered subcutaneously in a dosage of 3.6 mg every 21 to 28 days.

Buserelin may be administered intranasally or subcutaneously in a dosage of 100 to 1000 mcg once every 8 to 24 hours, for example, 0.5 mL injected sub-cutaneously 3 times a day at 8 hourly intervals for a period of 7 days.

Leuprolide may be administered subcutaneously or intramuscularly in a dosage of 1 to 30 mg every 1 to 120 days, for example, 7.5 mg via injection every month.

DES (diethylstilbestrol) may be administered orally in a dosage of 1 to 3 mg once or twice a day.

Cyproterone acetate may be administered orally or via intramuscular injection in a dosage of 100 to 300 mg from 1 to 4 times per day or week.

Mitoxantrone may be administered in a dosage of 10 to 15 mg/m² via IV infusion over 5 to 60 minutes every 14 to 28 days, for example, 12 to 14 mg/m² every 21 days.

Hydrocortisone may be administered orally in a dosage of 1 to 100 mg once or twice a day.

Estramustine may be administered orally in a dosage of 10 to 200 mg once or twice a day, for example, 10 to 14 mg per kg body mass, administered in 3 divided doses.

Prednisone may be administered orally in a dosage of 10 to 200 mg once or twice a day.

The method of the invention is useful where the cancer is characterized by amplification of the erbB gene, an overexpression of the erbB protein, or both. In one aspect, the erbB gene, the erbB protein, or both, are erbB2. The overexpression can be characterized by a +2 or +3 level. Any method standard in the art can used to measure the levels of amplification or overexpression. For example, the amplification can be measured by fluorescence in situ hybridization (FISH). An advantageous method is described by Coussens et al. Science 230, 1132 (1032). Overexpression can be measured by immunohistochemistry (IHC). An advantageous method is also described by Coussens et al. Id. Alternatively, the level of overexpression of erbB is inferred from clinical observations, without use of explicit measurement by IHC, but based rather on the patient history, the physical diagnosis or other elements of the diagnosis.

In one aspect of the method of the invention, the compound of formula 1 is administered at least about daily. In another aspect, the compound of formula 1 is administered at least about twice daily. The therapeutically effective amount of the compound of formula 1 can be about 25 mg/kg/day. In another aspect, the therapeutically effective amount of the compound of formula 1 is about 50 mg/kg/day.

The compound of formula 1 can be administered orally, buccally, sublingually, vaginally, intraduodenally, parenterally, topically, or rectally. The formulation will preferably be adapted to the particular mode of administration. The antibody can be administered substantially simultaneously with the compound of formula 1.

The method of the invention is applicable to a human. Non-humans can also be treated. For example, the mammal can be a horse.

The method of the invention is useful for administration to female mammals. The method can also be useful for males. The mammal can be an adult. In another aspect, infants, children, adolescents or the elderly can be treated with the methods of the invention.

The methods of the invention are applicable to a wide variety of abnormal cell growth conditions. In one aspect, the methods and kits are advantageously applied to cancers. The cancer can be selected from the group consisting of: lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. Other cancers can also be susceptible to treatment with the methods of the invention. In one aspect, the cancer is selected from the group consisting of ovarian cancer and breast cancer. In another aspect, the cancer is breast cancer.

The method of the invention is also applicable to adjuvant therapy, for example, in which the mammal, has received or is receiving a course of chemotherapeutic agents. In such an aspect, the remaining cancer may be a minimal residual disease. In another aspect, the method of the invention can be applied as a prophylactic measure. Thus, for example, the method can be applied to a mammal in cancer remission, in which no measurable disease can be detected.

In one aspect of the methods of the invention, the amount of the antibody to erbB protein is at least sufficient to produce therapeutic synergy. In consequence, the combination of the steps of the method of the invention is an improved treatment of a cancer when compared to either alone.

The invention also comprises a kit comprising: (a) an agent of formula 1, as described above, and (b) written instructions packaged with (a), for simultaneous or sequential administration with an additional therapeutic agent for the treatment of a cancer. Thus, the written instructions can elaborate and qualify the modes of administration.

In one aspect of the kit, the written instructions specify administration of 5-fluoruracil, bevacizumab, bicalutamide, buserelin, carboplatin, cetuximab, cisplatin, CP-547,632, CP-751,871, ticilimumab (CP-675,206), cyproterone acetate, DES (diethylstilbestrol), dexamethasone, docetaxel, doxorubicin, epirubicin, erlotinib, estramustine, exemestane, flutamide, gefitinib, gemcitabine, goserelin, hydrocortisone, irinotecan (oral or IV), ixabepilone (BMS-247550) ketoconazole, letrozole, leuprolide, mitoxantrone, nilutamine, oxaliplatin, paclitaxel, pertuzumab, prednisone, PROMUNE™ (CP-7909, PF-3512676), SU-11248, tamoxifen, and vinorelbine or combinations thereof. Moreover, the kit can comprise a fluid for reconstituting the additional agent, if supplied in the dry state. In another aspect, the written instruction specifies administration of E-2-Methoxy-N-(3-{4-[3-methyl-4-(6-methyl-pyridin-3-yloxy)-phenylamino]-quinazolin-6-yl}-allyl)-acetamide. In yet another aspect, the kit further comprises E-2-Methoxy-N-(3-{4-[3-methyl-4-(6-methyl-pyridin-3-yloxy)-phenylamino]-quinazolin-6-yl}-allyl)-acetamide.

The compounds of the present invention are potent inhibitors of the erbB family of oncogenic and protooncogenic protein tyrosine kinases, in particular erbB2, and thus are all adapted to therapeutic use as antiproliferative agents (eq., anticancer) in mammals, particularly in humans. In particular, the compounds of the present invention are useful in the prevention and treatment of a variety of human hyperproliferative disorders such as malignant and benign tumors of the, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that the methods and kits of the present invention may be effective against a range of leukemias and lymphoid malignancies.

The compounds of the present invention may also be useful in the treatment of additional disorders in which aberrant expression ligand/receptor interactions or activation or signaling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signaling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The in vitro activity of the compounds of formula 1 may be determined by the following procedure.

The c-erbB2 kinase assay is similar to that described previously in Schrang et. al. Anal. Biochem. 211, 1993, p 233-239. Nunc MaxiSorp 96-well plates are coated by incubation overnight at 37° C. with 100 mL per well of 0.25 mg/mL Poly (Glu, Tyr) 4:1 (PGT) (Sigma Chemical Co., St. Louis, Mo.) in PBS (phosphate buffered saline). Excess PGT is removed by aspiration, and the plate is washed three times with wash buffer (0.1% Tween 20 in PBS). The kinase reaction is performed in 50 mL of 50 mM HEPES (pH 7.5) containing 125 mM sodium chloride, 10 mM magnesium chloride, 0.1 mM sodium orthovanadate, 1 mM ATP, 0.48 mg/mL (24 ng/well) c-erbB2 intracellular domain. The intracellular domain of the erbB2 tyrosine kinase (amino acids 674-1255) is expressed as a GST fusion protein in Baculovirus and purified by binding to and elution from glutathionecoated beads. The compound in DMSO (dimethylsulfoxide) is added to give a final DMSO concentration of about 2.5%. Phosphorylation was initiated by addition of ATP (adenosine triphosphate) and proceeded for 6 minutes at room temperature, with constant shaking. The kinase reaction is terminated by aspiration of the reaction mixture and subsequent washing with wash buffer (see above). Phosphorylated PGT is measured by 25 minutes of incubation with 50 mL per well HRP-conjugated PY54 (Oncogene Science Inc. Uniondale, N.Y.) antiphosphotyrosine antibody, diluted to 0.2 mg/mL in blocking buffer (3% BSA and 0.05% Tween 20 in PBS). Antibody is removed by aspiration, and the plate is washed 4 times with wash buffer. The colorimetric signal is developed by addition of TMB Microwell Peroxidase Substrate (Kirkegaard and Perry, Gaithersburg, Md.), 50 mL per well, and stopped by the addition of 0.09 M sulfuric acid, 50 mL per well. Phosphotyrosine is estimated by measurement of absorbance at 450 nm. The signal for controls is typically 0.6-1.2 absorbance units, with essentially no background in wells without the PGT substrate and is proportional to the time of incubation for 10 minutes. Inhibitors were identified by reduction of signal relative to wells without inhibitor and IC₅₀ values corresponding to the concentration of compound required for 50% inhibition are determined. The compounds exemplified herein which correspond to formula 1 have IC50 values of <10 μM against erbB2 kinase.

The activity of the compounds of formula 1, in vivo, can be determined by the amount of inhibition of tumor growth by a test compound relative to a control. The tumor growth inhibitory effects of various compounds are measured according to the method of Corbett T. H., et al., “Tumor Induction Relationships in Development of Transplantable Cancers of the Colon in Mice for Chemotherapy Assays, with a Note on Carcinogen Structure”, Cancer Res., 35, 2434-2439 (1975) and Corbett T. H., et al., “A Mouse Colon-tumor Model for Experimental Therapy”, Cancer Chemother. Rep. (Part 2)”, 5, 169-186 (1975), with slight modifications. Tumors are induced in the left flank by subcutaneous (sc) injection of 5 million log phase cultured tumor cells (BT-474 human breast adenocarcinoma) suspended in Matrigel (1:1 in PBS). After sufficient time has elapsed for the tumors to become palpable (˜120 mm³ in size the test animals (athymic female mice) are treated with vehicles (0.5% methyl cellulose 10 ml/kg PO QD, PBS 5 ml/kg IP twice weekly or both), test compound (agent 182 formulated at a concentration of 10 to 15 mg/ml in 0.5% methyl cellulose, 25 or 50 mg/kg PO QD), Herceptin alone (0.1 or 0.3 mg/kg IP twice weekly) or both agent 182 and Herceptin (Table 1) for 28 consecutive days. In order to determine an anti-tumor effect, the tumor is measured in millimeters with a Vernier caliper across two diameters and the tumor size (mm³) is calculated using the formula: Tumor size (mm³)=(length×[width]2)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition (%)=(TuW_{control}−TuWₜₑₛₜ)/TuW_{control}×100%. The flank site of tumor implantation provides reproducible dose/response effects for a variety of chemotherapeutic agents, and the method of measurement (tumor diameter) is a reliable method for assessing tumor growth rates.

Administration of the compounds of the combination of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active small molecule compound (or ligand) administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

This invention also relates to a method for the treatment of abnormal cell growth in a mammal which comprises administering to said mammal an amount of a compound of formula 1, or a pharmaceutically acceptable salt, solvate or prodrug thereof, that is effective in treating abnormal cell growth in combination with an additional therapeutic agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a method for the treatment of a disorder associated with angiogenesis in a mammal, including a human, comprising administering to said mammal an amount of a compound of the formula 1, as defined above, or a pharmaceutically acceptable salt, solvate or prodrug thereof, that is effective in treating said disorder in combination with an additional therapeutic agent. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of formula 1, or a pharmaceutically acceptable salt, solvate or prodrug thereof, in combination with an additional therapeutic agent, and an amount of one or more substances selected from anti-angiogenesis agents, signal transduction inhibitors, and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of formula 1 in the methods and pharmaceutical compositions described herein. Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R,3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R,3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

VEGF inhibitors, for example, SU-11248, SU-5416 and SU-6668 (Sugen Inc. of South San Francisco, Calif., USA), can also be combined with a compound of formula 1. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome).

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent application Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

A compound of formula 1 may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

The combination of a compound of formula I and an additional therapeutic agent (herein referred to as the “combination of the invention” or the “combination of the present invention”) may be applied as a sole therapy or may involve one or more other anti-tumor substances, for example those selected from, for example, mitotic inhibitors, for example vinblastine; alkylating agents, for example cis-platin, oxaliplatin, carboplatin and cyclophosphamide; anti-metabolites, for example 5-fluorouracil, capecitabine, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide).

The combination of the invention may be used alone or in combination with one or more of a variety of anti-cancer agents or supportive care agents. For example, the combination of the present invention may be used with cytotoxic agents, e.g., one or more selected from the group consisting of a camptothecin, irinotecan HCl (Camptosar), edotecarin, SU-11248, epirubicin (Ellence), docetaxel (Taxotere), paclitaxel, rituximab (Rituxan) bevacizumab (Avastin), imatinib mesylate (Gleevac), Erbitux, gefitinib (Iressa), and combinations thereof. The invention also contemplates the use of the combination of the present invention together with hormonal therapy, e.g., exemestane (Aromasin), Lupron, anastrozole (Arimidex), tamoxifen citrate (Nolvadex), Trelstar, and combinations thereof. Further, the invention provides a combination of an anti-erbB2 antibody and a compound of formula I with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The combination of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the combination of the invention.

Alkylating agents include, but are not limited to, nitrogen mustard N-oxide, cyclophosphamide, ifosfamide, melphalan, busulfan, mitobronitol, carboquone, thiotepa, ranimustine, nimustine, or temozolomide;

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, gemcitabine, or fludarabine;

Antibiotics include but are not limited to, actinomycin D, doxorubicin, daunorubicin, neocarzinostatin, bleomycin, peplomycin, mitomycin C, aclarubicin, pirarubicin, epirubicin, zinostatin, stimalamer, or idarubicin;

Plant-derived antitumor agents include but are not limited to, vincristine, vinblastine, vindeshine, etoposide, sobuzoxane, docetaxel, paclitaxel, or vinorelbine;

Platinum-coordinated compounds include but are not limited to, cisplatin, carboplatin, nedaplatin, or oxaliplatin;

Camptothecin derivatives include but are not limited to camptothecin, 10-hydroxycamptothecin, 9-aminocamptothecin, irinotecan, SN-38, edotecarin, and topotecan;

Tyrosine kinase inhibitors are Iressa or SU5416;

Antibodies include Iressa, Erbitux, Avastin, or Rituximab;

Interferons include interferon alpha, interferon alpha-2a, interferon, alpha-2b, interferon beta, interferon gamma-1a or interferon gamma-n1;

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, or ubenimex; and

Other antitumor agents include mitoxantrone, I-asparaginase, procarbazine, dacarbazine, hydroxycarbamide, pentostatin, or tretinoin.

The examples, results, and preparations provided below further illustrate and exemplify the methods, kits, and compounds of the present invention, and the methods of preparing the compounds of formula 1. It is to be understood that the scope of the present invention is not limited in any way by the scope of the following examples, results, and preparations.

The small molecule ligands of the invention can be prepared according to the following information. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

### Method A: Synthesis of [3-Methyl-4-(Pyridin-3-yloxy)-phenyl]-(6-piperidin-4-ylethynyl-quinazolin-4-yl)-amine (1)

4-(4-Chloro-quinazolin-6-ylethynyl)-piperidine-1-carboxylic acid tert-butyl ester: A mixture of 4-ethynyl-piperidine-1-carboxylic acid tert-butyl ester (1.12 g, 5.35 mmol), 4-chloro-6-iodoquinazoline (1.35 g, 4.65 mmol), dichlorobis(triphenylphosphine) palladium(II) (0.16 g, 0.23 mmol), copper(I) iodide (0.044 g, 0.23 mmol), and diisopropylamine (0.47 g, 4.65 mmol) in anhydrous THF (20 mL) was stirred at room temperature under nitrogen for 2 hours. After concentration, the residue was dissolved in CH2Cl2 (100 mL), washed with aqueous NH4Cl and brine, dried over sodium sulfate, and concentrated to give the crude product as brown oil. Purification by silica gel column using 20% EtOAc in hexane afforded 1.63 g (94%) of the title compound as a sticky, yellow oil: 1H NMR (CDCl3) δ 1.45 (s, 9H), 1.67-1.75 (m, 2H), 1.87-1.92 (m, 2H), 2.84 (m, 1H), 3.20-3.26 (m, 2H), 3.78 (br d, 2H), 7.88 (dd, 1H), 7.97 (d, 1H), 8.26 (d, 1H), 9.00 (s, 1H).

[3-Methyl-4-(pyridin-3-yloxy)-phenyl]-(6-piperidin-4-ylethynyl-quinazolin-4-yl)-amine: 4-(4-Chloro-quinazolin-6-ylethynyl)-piperidine-1-carboxylic acid tert-butyl ester (80 mg, 0.21 mmol) and 3-Methyl-4-(pyridin-3-yloxy)-phenylamine (43 mg, 0.21 mmol) were mixed together in tert-butanol (1 mL) and dichloroethane (1 mL) and heated in a sealed vial at 90° C. for 20 minutes. The reaction was cooled down and HCl (gas) was bubbled through for 5 minutes. EtOAC was then added whereupon yellow precipitation occurred. The precipitate was collected and dried to afford the desired product [3-Methyl-4-(pyridin-3-yloxy)-phenyl]-(6-piperidin-4-ylethynyl-quinazolin-4-yl)-amine as a yellow solid (96 mg, 95%). ¹H NMR (CDCl₃) δ 2.01 ((m, 2H), 2.22 (m, 2H), 2.35(s, 3H), 3.20 (m, 2H), 3.45(m, 2H), 7.28 (d, 1H, J=8.7 Hz), 7.75(dd, 3H, J1=8.7, J2=8.7 Hz), 8.06 (dd, J=8.7), 8.10 (dd, J1=J2=8.7 Hz), 8.17 (m, 1 H), 8.60 (d, 1H, J=5.4 Hz), 8.80 (s, 1H), 8.89 (s, 1H). MS: M+1, 436.6.

### Method B: Synthesis of 2-Chloro-N-(3-{4-[3-methyl-4-(Pyridin-3-yloxy)-phenylamino]-quinazolin-6-yl}-prop-2-ynyl)-acetamide (2)

2-Chloro-N-[3-(4-chloro-quinazolin-6-yl)-prop-2-ynyl]-acetamide: 2-Chloro-N-prop-2-ynyl-acetamide (385 mg; 2.93 mmol) and 4-chloro-6-iodoquinazoline (850 mg; 1 equiv.) were dissolved in dry THF and diisopropylamine (296 mg; 0.41 mL; 1 equiv.). To this mixture was added 0.04 equivalents of copper iodide (22 mg) and Pd(PPh₃)₂Cl₂ (82 mg). The reaction was stirred at room temperature under a nitrogen atmosphere overnight (˜20 hrs). The solvent was then removed in vacuo and the residue dissolved in CH₂Cl₂. This solution was transferred to a separatory funnel and washed with 1× saturated NH₄Cl, brine, dried over Na₂SO₄ and the solvent removed in vacuo. The product was purified by silica gel chromatography eluting with 1:1 Hexanes/EtOAc and collecting fractions with an Rf=0.25. 2-Chloro-N-[3-(4-chloro-quinazolin-6-yl)-prop-2-ynyl]-acetamide was obtained as an off white solid (454 mg; 53%). ¹H NMR (400 MHz; CDCl₃) δ 4.12 (2H, s), 4.40 (2H, d, J=5.2 Hz), 7.91-7.93 (1H, dd, J=2, 6.8 Hz), 8.00 (1H, d, J=8.4 Hz), 8.34 (1H, d, J=1.6 Hz), 9.03 (1H, s). Irms (M+): 294.0, 296.0, 298.1.

2-Chloro-N-(3-{4-[3-methyl-4-(pyridin-3-yloxy)-phenylamino]-quinazolin-6-yl}-prop-2-ynyl)-acetamide: A mixture of 2-Chloro-N-[3-(4-chloro-quinazolin-6-yl)-prop-2-ynyl]-acetamide (0.90 g, 3.05 mmol) and 3-Methyl-4-(pyridin-3-yloxy)-phenylamine (0.61 g, 3.05 mmol) in ^{t}BuOH/DCE (5.0/5.0 mL) was refluxed under nitrogen for 40 minutes and concentrated. The residue was dissolved in MeOH (2.0 mL) and added to EtOAc with vigorous stirring to precipitate the HCl salt product as tan solid which was collected by vacuum-filtration, rinsed with EtOAc, and further dried to give 1.24 g (82%) of 2-Chloro-N-(3-{4-[3-methyl-4-(pyridin-3-yloxy)-phenylamino]-quinazolin-6-yl-prop-2-ynyl)-acetamide: ¹H NMR (CD₃OD) δ 2.27 (s, 3H), 4.09 (s, 2H), 4.29 (s, 2H), 7.07 (d, 1H), 7.51 (m, 2H), 7.60 (d, 1H), 7.70 (s, 1H), 7.78 (d, 1H), 8.05 (d, 1H), 8.32 (m, 2H), 8.67 (s, 1H), 8.75 (s, 1H); MS m/z (MH⁺) 458.0.

### Method C: Synthesis of 2-Dimethylamino-N-(3-(4-[3-methyl-4-(Pyridin-3-yloxy)-phenylamino]-quinazolin-6-yl}-prop-2-ynyl)-acetamide (3)

2-Dimethylamino-N-(3-{4-[3-methyl-4-(pyridin-3-yloxy)-phenylamino]-quinazolin-6-yl}-prop-2-ynyl)-acetamide: To a solution of 2-Chloro-N-(3-{4-[3-methyl-4-(pyridin-3-yloxy)-phenylamino]-quinazolin-6-yl}-prop-2-ynyl)-acetamide (99 mg, 0.20 mmol) in MeOH (5 mL) was added a solution dimethylamine in THF (2 mL, 4.0 mmol). The resulting solution was refluxed under nitrogen for 1 hour. After concentration, the residue was further dried, dissolved in MeOH (1.0 mL), and treated with HCl gas for 3 minutes. The resulting solution was added to EtOAc with vigorous stirring to precipitate the HCl salt product as yellow solid which was collected by vacuum-filtration, rinsed with EtOAc, and further dried to give 110 mg (99%) of the title compound. ¹H NMR (CD₃OD) δ 2.30 (s, 3H), 2.96 (s, 6H), 4.03 (s, 2H), 4.37 (s, 2H), 7.27 (d, 1H), 7.72 (dt, 1H), 7.81(m, 1H), 7.84 (d, 1H), 8.03 (dd, 1H), 8.06 (d, 1H), 8.13 (dd, 1H), 8.59 (d, 1H), 8.68 (s, 1H), 8.81 (s, 1H), 8.84 (s, 1H); MS m/z (MH⁺) 467.3.

### Method D: Synthesis of 1-(3-{4-[3-Chloro-4-(6-methyl-pyridin-3-yloxy)-phenylamino]-quinazolin-6-yl-prop-2-ynyl)-3-methyl-urea (4)

1-(3-{4-[3-Chloro-4-(6-methyl-pyridin-3-yloxy)-phenylamino]-quinazolin-6-yl}-prop-2-ynyl)-3-methyl-urea: A mixture of (3-{4-[3-Chloro-4-(6-methyl-pyridin-3-yloxy)-phenylamino]-quinazolin-6-yl}-prop-2-ynyl)-carbamic acid phenyl ester (0.1 g, 0.18 mmol) prepared by Method B, methyl amine (2.0M methanol solution, 1 mL, 2 mmol) and DMSO (0.5 mL) was stirred at 80° C. overnight. The solvents were removed under vacuum (GeneVac HT-8) and the residue was re-dissolved in MeOH (˜1 mL). HCl gas was bubbled through the solution and EtOAc resulting in precipitation of the desired product. The title compound (80 mg, 90% yield) was obtained by filtration as a yellow solid. ¹HNMR (400 MHz, CD₃OD) δ 2.72 (3H,s), 2.76 (3H, s), 4.19 (2H, s), 7.49 (1H, d, J=9 Hz), 7.84 (1H, d, J=2 Hz), 7.86 (1H, d, J=2 Hz), 7.92 (1H, d, J=9 Hz), 8.12 (2H, m, J=2 Hz), 8.16 (1H, d, J=2.4 Hz), 8.60 (1H, d, J=3.2 Hz), 8.74 (1H, d, J=1.2 Hz), 8.87 (1H, s). LRMS (M⁺): 473.0, 475.0, 476.0.

### Method E: Synthesis of 3-{4-[3-Methyl-4-(pyridin-3-yloxy)-phenylamino]-quinazolin-6-yl-prop-2-en-1-ol (5)

3-{4-[3-Methyl-4-(pyridin-3-yloxy)-phenylamino]-quinazolin-6-yl}-prop-2-en-1-ol. To a solution of 0.56 g (1.47 mmol) of 3-{4-[3-methyl-4-(pyridin-3-yloxy)-phenylamino]-quinazolin-6-yl}-prop-2-yn-1-ol (prepared by Method B) in 6 mL of dry tetrahydrofuran at 0° C. was added 0.73 mL of a 65% weight toluene solution of sodium bis(2-methoxyethoxy)aluminum hydride (Red-Al, 2.35 mmol) in 1 mL of THF. The reaction was stirred at room temperature for 3 hours. Upon recooling to 0° C. an additional 0.73 mL of the Red-Al solution in 1 mL of THF was added. After stirring for 1 hour at room temperature, the mixture was quenched with the dropwise addition of 10% aqueous potassium carbonate and extracted with ethyl acetate. The organic extracts were dried over sodium sulfate, filtered and evaporated to give 650 mg. Chromatography on 90 g silica gel, eluting with 96:4:0.1 chloroform/methanol/concentrated ammonium hydroxide afforded 268 mg of the title compound. ¹H NMR (d₆ DMSO): δ 9.79 (s, 1), 8.57 (m, 2), 8.35 (m, 2), 8.01 (m, 1), 7.80 (m, 3), 7.41 (m, 1), 7.29 (m, 1), 7.07 (d, J=8.7 Hz, 1), 6.77 (d, J=16.2 Hz, 1), 6.67 (m, 1), 5.04 (t, J=5.6 Hz, 1), 4.23 (m, 2), 2.23 (s, 3).

### Method F: Synthesis of [3-Methyl-4-(pyridin-3-yloxy)-phenyl]-[6-(3-morpholin-4-yl-propenyl)-quinazolin-4-yl]-amine (6)

[3-Methyl-4-(pyridin-3-yloxy)-phenyl]-[6-(3-morpholin-4-yl-propenyl)-quinazolin-4-yl]-amine. To a suspension of 0.035 g (0.091 mmol) of 3-{4-[3-methyl-4-(pyridin-3-yloxy)-phenylamino]-quinazolin-6-yl}-prop-2-en-1-ol in 0.5 mL of methylene chloride and 1 mL of ethylene dichloride was added 1 mL of thionyl chloride. The reaction was heated at 100° C. for 1 hour and the solvents were evaporated to provide [6-(3-chloro-propenyl)-quinazolin-4-yl]-[3-methyl-4-(pyridin-3-yloxy)-phenyl]-amine [MS: M⁺ 403.1] which was dissolved in THF and used directly in the next reaction. To the solution of [6-(3-chloro-propenyl)-quinazolin-4-yl]-[3-methyl-4-(pyridin-3-yloxy)-phenyl]-amine was added 0.10 mL of morpholine and 0.044 mL of triethylamine. The mixture was heated at 85° C. for 16 hours, cooled to room temperature, and partitioned between 10% aqueous potassium carbonate and ethyl acetate. The aqueous layer was further extracted with ethyl acetate and the combined organics were dried and evaporated to yield 57 mg of material. The product was purified on a silica gel prep plate, eluting with 96:4:0.1 chloroform/methanol/concentrated ammonium hydroxide to afford 26 mg of the title compound; ¹H NMR (CDCl₃): δ 8.71 (s, 1), 8.33 (m, 2), 7.94 (s, 1), 7.80 (m, 2), 7.69 (s, 1), 7.58 (m, 1), 7.20 (m, 1), 6.94 (d, J=8.7 Hz, 1), 6.68 (d, J=15.8 Hz, 1), 6.46 (m, 1), 3.79 (m, 4), 3.26(m, 2), 2.63 (m, 4), 2.25 (s, 3).

### Method G: Synthesis of E-N-(3-{4-[3-Chloro-4-(6-methyl-pyridin-3-yloxy)-phenylamino]-quinazolin-6-yl}-allyl)-acetamide (7)

E-(3-{4-[3-chloro-4-(6-methyl-pyridin-3-yloxy)-phenylamino]-quinazolin-6-yl}-allyl)-carbamic acid tert-butyl ester: To a solution of 7.53 mL of a 65% weight toluene solution of sodium bis(2-methoxyethoxy)aluminum hydride (Red-Al, 24.2 mmol) in 90 mL of tetrahydrofuran at 0° C. was added 5.0 g of (3-{4-[3-chloro-4-(6-methyl-pyridin-3-yloxy)-phenylamino]-quinazolin-6-yl}-prop-2-ynyl)-carbamic acid tert-butyl ester as a solid. The reaction was stirred at 0° C. for 2 hours, quenched with 10% aqueous potassium carbonate and extracted with ethyl acetate. The combined organics were dried and evaporated. The crude material was purified on 115 g of silica gel, eluting with 80% ethyl acetate/hexanes to afford 4.42 g of E-(3-{4-[3-chloro-4-(6-methyl-pyridin-3-yloxy)-phenylamino]-quinazolin-6-yl}-allyl)-carbamic acid tert-butyl ester. ¹H NMR (CDCl₃): δ 8.66 (s, 1), 8.24 (m, 1), 8.03 (m, 2), 7.77-7.65 (m, 3), 7.13 (m, 2), 6.97 (d, J=8.7 Hz, 1), 6.54 (d, 1), 6.35 (m, 1), 4.9 (m, 1), 3.90 (m, 2), 2.52 (s, 3), 1.46 (s, 9).

E-[6-(3-amino-propenyl)-quinazolin-4-yl]-[3-chloro-4-(6-methyl-pyridin-3-yloxy)-phenyl]-amine. To a solution of 4.42 g of E-(3-{4-[3-chloro-4-(6-methyl-pyridin-3-yloxy)-phenylamino]-quinazolin-6-yl}-allyl)-carbamic acid tert-butyl ester in 21 mL of tetrahydrofuran was added 21 mL of 2 N hydrochloric acid. The mixture was heated at 60° C. for 3 hours, cooled to room temperature and basified with 10% aqueous potassium carbonate. Methylene chloride was added to the aqueous mixture and a solid precipitated. The solid was filtered and dried to yield 2.98 g of E-[6-(3-amino-propenyl)-quinazolin-4-yl]-[3-chloro-4-(6-methyl-pyridin-3-yloxy)-phenyl]-amine. ¹H NMR (d₆ DMSO): δ 8.62 (s, 1), 8.53 (m, 1), 8.26 (m, 2), 7.99 (m, 1), 7.89 (m, 1), 7.77 (m, 1), 7.30 (m, 3), 6.67 (m, 2), 3.44 (m, 2), 2.47 (s, 3).

E-N-(3-{4-[3-Chloro-4-(6-methyl-pyridin-3-yloxy)-phenylamino]-quinazolin-6-yl}-allyl)-acetamide. A mixture of 14.4 μL (0.25 mmol) of acetic acid and 40.3 mg (0.33 mmol) of dicyclohexylcarbodiimide in 2 mL of methylene chloride were stirred for 10 minutes and treated with 100.3 mg of E-[6-(3-amino-propenyl)-quinazolin-4-yl]-[3-chloro-4-(6-methyl-pyridin-3-yloxy)-phenyl]-amine. The reaction was allowed to stir at room temperature overnight. The precipitate which formed was filtered and chromatographed on silica gel, eluting with 6-10% methanol/chloroform to afford 106 mg of the title compound; mp 254-256° C.; ¹H NMR (d₆ DMSO): δ 9.88 (s, 1), 8.58 (s, 1), 8.48 (m, 1), 8.20 (m, 3), 7.95 (m, 1), 7.83 (m, 1), 7.71(d, J=8.7 Hz, 1), 7.24 (m, 2), 7.19 (d, J=8.7 Hz, 1), 6.61 (d, J=16.2 Hz, 1), 6.48 (m, 1), 3.90 (m, 2).

### Method H: E-2S-Methoxymethyl-pyrrolidine-1-carboxylic acid (3-{4-[3-methyl-4-(6-methyl-pyridin-3-yloxy)-phenylamino]-quinazolin-6-yl}-allyl)-amide (8)

To a stirred solution of 0.125 g (0.31 mmol) of E-[6-(3-amino-propenyl)-quinazolin-4-yl]-[3-methyl-4-(6-methyl-pyridin-3-yloxy)-phenyl]-amine (prepared according to method G) in 1 mL of dichloromethane at 0° C. was added 60.3 μL (0.34 mmol) of Hunig's base followed by dropwise addition of a solution of 48.2 uL (0.34 mmol) of 4-chlorophenyl chloroformate in 1 mL of dichloromethane. The reaction was stirred 30 minutes and evaporated under reduced pressure. The residue was dissolved in 2 mL of dimethyl sulfoxide and 123 μL (0.94 mmol) of (S)-(+)-2-(methoxymethyl)-pyrrolidine was added neat. The reaction was stirred for 3 hours at room temperature. The reaction was quenched into 10% potassium carbonate and extracted with ethyl acetate. The organic layer was washed several times with water and twice with brine. The organic layer was dried over sodium sulfate and reduced to yield the crude material. This material was purified over 90 g of silica gel using 96:4:0.1 chloroform:methanol:ammonium hydroxide as eluent to yield 75 mg (0.14 mmol) of the title compound. ¹HNMR (d₆ DMSO): δ 9.83 (s, 1), 8.56 (s, 2), 8.21 (d, 1), 7.95 (d, 1), 7.80 (d, 1), 7.50 (d, 1), 7.25 (m, 2), 7.01 (d, 1), 6.63 (d, 1), 6.53 (m, 1), 3.95 (m, 2), 3.40 (dd, 1), 3.28 (s, 3), 2.49 (s, 3), 2.24 (s, 3), 1.85 (m, 4).

### Method I: E-2-Hydroxy-N-(3-{4-[3-methyl-4-(6-methyl-pyridin-3-yloxy)-phenylamino]-quinazolin-6-yl}-allyl)-isobutyramide (9)

To a solution of 0.170 g (0.42 mmol) of E-[6-(3-amino-propenyl)-quinazolin-4-yl]-[3-methyl-4-(6-methyl-pyridin-3-yloxy)-phenyl]-amine (prepared according to method G) in 1 mL of dichloromethane at 0° C. was added 65 μL (0.47 mmol) of triethylamine followed by a solution of 65 μL (0.45 mmol) of 2-acetoxyisobutyryl chloride in 1 mL of dichloromethane. The reaction was stirred at 0° C. for 1 hour. The mixture was quenched with a dropwise addition of 10% potassium carbonate. The aqueous layer was extracted with dichloromethane and the combined organics were washed with brine, dried over sodium sulfate and evaporated. The crude material was purified on 90 g of silica gel eluting with 96:4:0.1 chloroform/methanol/ammonium hydroxide to afford 2-acetoxy-N-(3-{4-[3-methyl-4-(6-methyl-pyridin-3-yloxy)-phenylamino]-quinazolin-6-yl}-allyl)-isobutyramide. A solution of this material in 2 mL of methanol was treated dropwise with a solution of 41 mg (3.02 mmol) of potassium carbonate in 0.5 mL of water. The solution was stirred at room temperature for 1 hour. The reaction was evaporated and the residue was partitioned between water and chloroform. The aqueous layer was extracted twice with chloroform and the combined organics were washed with brine, dried over sodium sulfate and evaporated to yield 100 mg of the title compound (47%). ¹HNMR (d₆ DMSO): δ 9.78 (s, 1), 8.50 (s, 1), 8.48 (s, 1), 8.15 (d, 1), 7.95 (m, 2), 7.65 (m, 3), 7.21 (m, 2), 6.96 (d, 1), 6.56 (dt, 1), 3.92 (t, 2), 2.46 (s, 3), 2.1.

The following examples were prepared using the methods described above.

TABLE 1

| Example No. | Name | Method | LRMS | HPLC RT |
|---|---|---|---|---|
| 1 | (±)-[3-Methyl-4-(pyridin-3-yloxy)- | A | 436.0 | 4.48 |
| | phenyl]-(6-piperidin-3-ylethynyl- | | | |
| | quinazolin-4-yl)-amine | | | |
| 2 | 1-(3-{4-[3-Chloro-4-(6-methyl-pyridin-3- | D | 499.0 | 5.74 |
| | yloxy)-phenylamino]-quinazolin-6-yl}- | | | |
| | prop-2-ynyl)-3-cyclopropyl-urea | | | |
| 3 | N-(3-{7-Chloro-4-[3-chloro-4-(6-methyl- | B | 492.0 | 6.07 |
| | pyridin-3-yloxy)-phenylamino]- | | | |
| | quinazolin-6-yl}-prop-2-ynyl)- | | | |
| | acetamide | | | |
| 4 | N-(3-{7-Chloro-4-[3-methyl-4-(6- | B | 472.2 | 5.79 |
| | methyl-pyridin-3-yloxy)-phenylamino]- | | | |
| | quinazolin-6-yl}-prop-2-ynyl)- | | | |
| | acetamide | | | |
| 5 | Exo-6-Hydroxymethyl-3-aza- | D | 555.0 | 5.19 |
| | bicyclo[3.1.0]hexane-3-carboxylic acid | | | |
| | (3-{4-[3-chloro-4-(6-methyl-pyridin-3- | | | |
| | yloxy)-phenylamino]-quinazolin-6-yl}- | | | |
| | prop-2-ynyl)-amide | | | |
| 6 | 1-(3-{4-[3-Chloro-4-(6-methyl-pyridin-3- | D | 505.0 | 5.65 |
| | yloxy)-phenylamino]-quinazolin-6-yl}- | | | |
| | prop-2-ynyl)-3-(2-fluoro-ethyl)-urea | | | |
| 7 | 1-(3-{4-[3-Chloro-4-(6-methyl-pyridin-3- | D | 503.0 | 4.98 |
| | yloxy)-phenylamino]-quinazolin-6-yl}- | | | |
| | prop-2-ynyl)-3-(2-hydroxy-ethyl)-urea | | | |
| 8 | 3-{4-[3-Methyl-4-(pyridin-3-yloxy)- | A | 452.0 | 4.01 |
| | phenylamino]-quinazolin-ylethynyl}- | | | |
| | piperidin-3-ol | | | |
| 9 | 2-(2-Hydroxy-ethylsulfanyl)-N-(3-{4-[3- | C | 500.0 | 4.87 |
| | methyl-4-(pyridin-3-yloxy)- | | | |
| | phenylamino]-quinazolin-6-yl}-prop-2- | | | |
| | ynyl)-acetamide | | | |
| 10 | N-(3-{4-[3-Chloro-4-(pyridin-3-yloxy)- | C | 520.0 | 5.15 |
| | phenylamino]-quinazolin-6-yl}-prop-2- | | | |
| | ynyl)-2-(2-hydroxy-ethylsulfanyl)- | | | |
| | acetamide | | | |
| 11 | (±)-[3-Methyl-4-(pyridin-3-yloxy)- | A | 438.0 | 4.29 |
| | phenyl]-(6-morpholin-2-ylethynyl- | | | |
| | quinazolin-4-yl)-amine | | | |
| 12 | 2-Cyano-N-(3-{4-[3-methyl-4-(pyridin- | B | 448.9 | 5.18 |
| | 3-yloxy)-phenylamino]-quinazolin-6-yl}- | | | |
| | prop-2-ynyl)-acetamide | | | |
| 13 | N-(3-{4-[3-Methyl-4-(pyridin-3-yloxy)- | B | 452.0 | 5.61 |
| | phenylamino]-quinazolin-6-yl}-prop-2- | | | |
| | ynyl)-butyramide | | | |
| 14 | Pentanoic acid (3-{4-[3-methyl-4- | B | 466.0 | 6.02 |
| | (pyridin-3-Ybloxy)-phenylamino]- | | | |
| | quinazolin-6-yl}-prop-2-ynyl)-amide | | | |
| 15 | 2-Methoxy-N-(3-{4-[3-methyl-4- | B | 454.0 | 5.24 |
| | (pyridin-3-yloxy)-phenylamino]- | | | |
| | quinazolin-6-yl}-prop-2-ynyl)- | | | |
| | acetamide | | | |
| 16 | N-(4-{4-[3-Methyl-4-(pyridin-3-yloxy)- | B | 438.1 | 5.11 |
| | phenylamino]-quinazolin-6-yl}-but-3- | | | |
| | ynyl)-acetamide | | | |
| 17 | [6-(4-Amino-but-1-ynyl)-quinazolin-4- | A | 396.1 | 4.04 |
| | yl]-[3-methyl-4-(pyridin-3-yloxy)- | | | |
| | phenyl]-amine | | | |
| 18 | N-(3-{4-[3-Methyl-4-(pyridin-3-yloxy)- | B | 470.2 | 5.50 |
| | phenylamino]-quinazolin-6-yl}-prop-2- | | | |
| | ynyl)-2-methylsulfanyl-acetamide | | | |
| 19 | 3-{4-[3-Methyl-4-(pyridin-3-yloxy)- | B | 383.0 | 4.97 |
| | phenylamino]-quinazolin-6-yl}-prop-2- | | | |
| | yn-1-ol | | | |
| 20 | [6-(3-Methoxy-prop-1-ynyl)-quinazolin- | B | 397.3 | 6.23 |
| | 4-yl]-[3-methyl-4-(pyridin-3-yloxy)- | | | |
| | phenyl]-amine | | | |
| 21 | 4-{4-[3-Methyl-4-(pyridin-3-yloxy)- | B | 397.1 | 5.17 |
| | phenylamino]-quinazolin-6-yl}-but-3- | | | |
| | yn-1-ol | | | |
| 22 | 2-Methyl-4-{4-[3-methyl-4-(pyridin-3- | B | 411.0 | 5.62 |
| | yloxy)-phenylamino]-quinazolin-6-yl}- | | | |
| | but-3-yn-2-ol | | | |
| 23 | (3-{4-[3-Methyl-4-(pyridin-3-yloxy)- | B | 440.3 | 5.61 |
| | phenylamino]-quinazolin-6-yl}-prop-2- | | | |
| | ynyl)-carbamic acid methyl ester | | | |
| 24 | N-(3-{4-[3-Methyl-4-(pyridin-3-yloxy)- | B | 460.0 | 5.38 |
| | phenylamino]-quinazolin-6-yl}-prop-2- | | | |
| | ynyl)-methanesulfonamide | | | |
| 25 | N-(3-{4-[3-Methyl-4-(pyridin-3-yloxy)- | B | 424.1 | 4.94 |
| | phenylamino]-quinazolin-6-yl}-prop-2- | | | |
| | ynyl)-acetamide | | | |
| 26 | [3-Methoxy-4-(pyridin-3-yloxy)-phenyl]- | A | 452.0 | 4.10 |
| | (6-piperidin-3-ylethynyl-quinazolin-4- | | | |
| | yl)-amine | | | |
| 27 | 2-Chloro-N-(3-{4-[3-methyl-4-(pyridin- | B | 458.0 | 5.52 |
| | 3-yloxy)-phenylamino]-quinazolin-6-yl}- | | | |
| | prop-2-ynyl)-acetamide | | | |
| 28 | 2-Methylamino-N-(3-{4-[3-methyl-4- | C | 453.1 | 4.08 |
| | (pyridin-3-yloxy)-phenylamino]- | | | |
| | quinazolin-6-yl}-prop-2-ynyl)- | | | |
| | acetamide | | | |
| 29 | 2-Dimethylamino-N-(3-{4-[3-methyl-4- | C | 467.3 | 4.15 |
| | (pyridin-3-yloxy)-phenylamino]- | | | |
| | quinazolin-6-yl}-prop-2-ynyl)- | | | |
| | acetamide | | | |
| 30 | (±)-(6-Piperidin-3-ylethynyl-quinazolin- | A | 422.1 | 4.13 |
| | 4-yl)-[4-(pyridin-3-yloxy)-phenyl]-amine | | | |
| 31 | [3-Methoxy-4-(pyridin-3-yloxy)-phenyl]- | A | 452.1 | 4.11 |
| | (6-piperidin-4-quinazolin-4-yl)-amine | | | |
| 32 | [3-Chloro-4-(pyridin-3-yloxy)-phenyl]- | A | 456.1 | 4.57 |
| | (6-piperidin-4-ylethynyl-quinazolin-4- | | | |
| | yl)-amine | | | |
| 33 | [3-Fluoro-4-(pyridin-3-yloxy)-phenyl]- | A | 440.1 | 4.38 |
| | (6-piperidin-4-ylethynyl-quinazolin-4- | | | |
| | yl)-amine | | | |
| 34 | (6-Piperidin-4-ylethynyl-quinazolin-4- | A | 422.1 | 4.11 |
| | yl)-[4-(pyridin-3-yloxy)-phenyl]-amine | | | |
| 35 | 2-Methoxy-N-(3-{4-[3-methoxy-4- | B | 470.3 | 4.87 |
| | (pyridin-3-yloxy)-phenylamino]- | | | |
| | quinazolin-6-yl}-prop-2-ynyl)- | | | |
| | acetamide | | | |
| 36 | N-(3-{4-[3-Chloro-4-(pyridin-3-yloxy)- | B | 474.2 | 5.48 |
| | phenylamino]-quinazolin-6-yl}-prop-2- | | | |
| | ynyl)-2-methoxy-acetamide | | | |
| 37 | N-(3-{4-[3-Fluoro-4-(pyridin-3-yloxy)- | B | 458.3 | 5.23 |
| | phenylamino]-quinazolin-6-yl}-prop-2- | | | |
| | ynyl)-2-methoxy-acetamide | | | |
| 38 | [3-Methyl-4-(pyridin-2-yloxy)-phenyl]- | A | 436.0 | 4.52 |
| | (6-piperidin-4-ylethynyl-quinazolin-4- | | | |
| | yl)-amine | | | |
| 39 | 2,2-Dimethyl-N-(3-{4-[3-methyl-4- | A | 452.3 | 5.60 |
| | (pyridin-3-yloxy)-phenylamino]- | | | |
| | quinazolin-6-yl}-prop-2-ynyl)- | | | |
| | propionamide | | | |
| 40 | N-(3-{4-[3-Methyl-4-(pyridin-3-yloxy)- | B | 466.3 | 6.01 |
| | phenylamino]-quinazolin-6-yl}-prop-2- | | | |
| | ynyl)-isobutyramide | | | |
| 41 | {6-[3-(2-Methoxy-ethoxy)-prop-1-ynyl]- | B | 441.1 | 6.11 |
| | quinazolin-4-yl}-[3-methyl-4-(pyridin-3- | | | |
| | yloxy)-phenyl]-amine | | | |
| 42 | 2-Diethylamino-N-(3-{4-[3-methyl-4- | C | 495.1 | 4.45 |
| | (pyridin-3-yloxy)-phenylamino]- | | | |
| | quinazolin-6-yl}-prop-2-ynyl)- | | | |
| | acetamide | | | |
| 43 | (±)-[3-Methyl-4-(2-methyl-pyridin-3- | A | 450.0 | 4.47 |
| | yloxy)-phenyl]-(6-piperidin-3-ylethynyl- | | | |
| | quinazolin-4-yl)-amine | | | |
| 44 | [3-Methyl-4-(2-methyl-pyridin-3-yloxy)- | A | 450.0 | 4.39 |
| | phenyl]-(6-piperidin-4-ylethynyl- | | | |
| | quinazolin-4-yl)-amine | | | |
| 45 | 2-Methoxy-N-(3-{4-[3-methyl-4-(2- | B | 468.0 | 5.33 |
| | methyl-pyridin-3-yloxy)-phenylamino]- | | | |
| | quinazolin-6-yl}-prop-2-ynyl)- | | | |
| | acetamide | | | |
| 46 | 2-(2-Methoxy-ethoxy)-N-(3-{4-[3- | B | 498.3 | 5.34 |
| | methyl-4-(pyridin-3-yloxy)- | | | |
| | phenylamino]-quinazolin-6-yl}-prop-2- | | | |
| | ynyl)-acetamide | | | |
| 47 | (±)-Tetrahydro-furan-2-carboxylic acid | B | 480.0 | 5.45 |
| | (3-{4-[3-methyl-4-(pyridin-3-yloxy)- | | | |
| | phenylamino]-quinazolin-6-yl}-prop-2- | | | |
| | ynyl)-amide | | | |
| 48 | (±)-4,4-Dimethyl-5-{4-[3-methyl-4- | B | 466.0 | 5.70 |
| | (pyridin-3-yloxy)-phenylamino]- | | | |
| | quinazolin-6-ylethynyl}-oxazolidin-2- | | | |
| | one | | | |
| 49 | {6-[4-(2-Methoxy-ethoxy)-but-1-ynyl]- | B | 455.3 | 6.23 |
| | quinazolin-4-yl}-[3-methyl-4-(pyridin-3- | | | |
| | yloxy)-phenyl]-amine | | | |
| 50 | 4-{4-[3-Methyl-4-(pyridin-3-yloxy)- | A | 452.0 | 3.82 |
| | phenylamino]-quinazolin-6-ylethynyl}- | | | |
| | piperidin-4-ol | | | |
| 51 | 1-Methyl-4-{4-[3-methyl-4-(pyridin-3- | B | 466.1 | 4.03 |
| | yloxy)-phenylamino]-quinazolin-6- | | | |
| | ylethynyl}-piperidin-4-ol | | | |
| 52 | (±)-[3-Methyl-4-(6-methyl-pyridin-3- | A | 450.0 | 4.52 |
| | yloxy)-phenyl]-(6-piperidin-3-ylethynyl- | | | |
| | quinazolin-4-yl)-amine | | | |
| 53 | [3-Methyl-4-(6-methyl-pyridin-3-yloxy)- | A | 450.0 | 4.49 |
| | phenyl]-(6-piperidin-4-ylethynyl- | | | |
| | quinazolin-4-yl)-amine | | | |
| 54 | 2-Methoxy-N-(3-{4-[3-methyl-4-(6- | B | 468.8 | 5.38 |
| | methyl-pyridin-3-yloxy)-phenylamino]- | | | |
| | quinazolin-6-yl}-prop-2-ynyl)- | | | |
| | acetamide | | | |
| 55 | [6-(4-Methoxy-but-1-ynyl)-quinazolin-4- | B | 411.2 | 6.30 |
| | yl]-[3-methyl-4-(pyridin-3-yloxy)- | | | |
| | phenyl]-amine | | | |
| 56 | (±)-4-(2-Chloro-pyridin-3-yloxy)-3- | A | 470.0 | 4.89 |
| | methyl-phenyl]-(6-piperidin-3-ylethynyl- | | | |
| | quinazolin-4-yl)-amine | | | |
| 57 | Cyclopropanecarboxylic acid (4-{4-[3- | B | 464.3 | 5.63 |
| | methyl-4-(pyridin-3-yloxy)- | | | |
| | phenylamino]-quinazolin-6-yl}-but-3- | | | |
| | ynyl)-amide | | | |
| 58 | [4-(2-Chloro-pyridin-3-yloxy)-3-methyl- | A | 470.0 | 4.86 |
| | phenyl]-(6-piperidin-4-ylethynyl- | | | |
| | quinazolin-4-yl)-amine | | | |
| 59 | N-(3-{4-[4-(2-Chloro-pyridin-3-yloxy)-3- | B | 488.0 | 5.84 |
| | methyl-phenylamino]-quinazolin-6-yl}- | | | |
| | prop-2-ynyl)-2-methoxy-acetamide | | | |
| 60 | N-(4-{4-[3-Methyl-4-(pyridin-3-yloxy)- | B | 484.2 | 5.64 |
| | phenylamino]-quinazolin-6-yl}-but-3- | | | |
| | ynyl)-2-methylsulfanyl-acetamide | | | |
| 61 | [3-Chloro-4-(pyridin-3-yloxy)-phenyl]- | B | 431.1 | 6.67 |
| | [6-(4-methoxy-but-1-ynyl)-quinazolin-4- | | | |
| | yl]-amine | | | |
| 62 | (±)-4-{4-[3-Methyl-4-(pyridin-3-yloxy)- | A | 413.1 | 4.31 |
| | phenylamino]-quinazolin-6-yl}-but-3- | | | |
| | yne-1,2-diol | | | |
| 63 | (±)-[3-Methyl-4-(pyridin-4-yloxy)- | A | 434.1 | 3.88 |
| | phenyl]-(6-piperidin-3-ylethynyl- | | | |
| | quinazolin-4-yl)-amine | | | |
| 64 | [3-Methyl-4-(pyridin-4-yloxy)-phenyl]- | A | 436.1 | 3.91 |
| | (6-piperidin-4-ylethynyl-quinazolin-4- | | | |
| | yl)-amine | | | |
| 65 | 2-Methoxy-N-(3-{4-[3-methyl-4- | B | 452.0 | 4.71 |
| | (pyridin-4-yloxy)-phenylamino]- | | | |
| | quinazolin-6-yl}-prop-2-ynyl)- | | | |
| | acetamide | | | |
| 66 | 2,2-Difluoro-N-(3-{4-[3-methyl-4- | B | 460.2 | 5.63 |
| | (pyridin-3-yloxy)-phenylamino]- | | | |
| | quinazolin-6-yl}-prop-2-ynyl)- | | | |
| | acetamide | | | |
| 67 | N-(3-{4-[3-Chloro-4-(pyridin-3-yloxy)- | B | 482.2, | 5.92 |
| | phenylamino]-quinazolin-6-yl}-prop-2- | | 480.1 | |
| | ynyl)-2,2-difluoro-acetamide | | | |
| 68 | R-Pyrrolidine-2-carboxylic acid (3-{4- | A | 479.1 | 4.22 |
| | [3-methyl-4-(pyridin-3-yloxy)- | | | |
| | phenylamino]-quinazolin-6-yl}-prop-2- | | | |
| | ynyl)-amide | | | |
| 69 | (±)-Tetrahydro-furan-3-carboxylic acid | B | 500.0 | 5.39 |
| | (3-{4-[3-chloro-4-(pyridin-3-yloxy)- | | | |
| | phenylamino]-quinazolin-6-yl}-prop-2- | | | |
| | ynyl)-amide | | | |
| 70 | Cyclopropanecarboxylic acid (4-{4-[3- | B | 484.0 | 5.92 |
| | chloro-4-(pyridin-3-yloxy)- | | | |
| | phenylamino]-quinazolin-6-yl}-but-3- | | | |
| | ynyl)-amide | | | |
| 71 | N-(4-{4-[3-Chloro-4-(pyridin-3-yloxy)- | B | 505.4 | 5.91 |
| | phenylamino]-quinazolin-6-yl}-but-3- | | | |
| | ynyl)-2-methylsulfanyl-acetamide | | | |
| 72 | 1-(3-{4-[3-Methyl-4-(pyridin-3-yloxy)- | D | 501.1 | 6.17 |
| | phenylamino]-quinazolin-6-yl}-prop-2- | | | |
| | ynyl)-3-phenyl-urea | | | |
| 73 | 1-Cyclohexyl-3-(3-{4-[3-methyl-4- | D | 507.2 | 6.24 |
| | (pyridin-3-yloxy)-phenylamino]- | | | |
| | quinazolin-6-yl}-prop-2-ynyl)-urea | | | |
| 74 | 1-(3-{4-[3-Chloro-4-(pyridin-3-yloxy)- | D | 528.1 | 6.49 |
| | phenylamino]-quinazolin-6-yl}-prop-2- | | | |
| | ynyl)-3-cyclohexyl-urea | | | |
| 75 | 2-Hydroxy-N-(4-{4-[3-methyl-4-(pyridin- | A | 454.2 | 4.78 |
| | 3-yloxy)-phenylamino]-quinazolin-6-yl}- | | | |
| | but-3-ynyl)-acetamide | | | |
| 76 | E-3-{4-[3-Methyl-4-(pyridin-3-yloxy)- | E | 385.1 | 4.71 |
| | phenylamino]-quinazolin-6-yl}-prop-2- | | | |
| | en-1-ol | | | |
| 77 | E-[3-Methyl-4-(pyridin-3-yloxy)-phenyl]- | F | 454.1 | 4.14 |
| | [6-(3-morpholin-4-yl-propenyl)- | | | |
| | quinazolin-4-yl]-amine | | | |
| 78 | 2-Methanesulfonyl-N-(3-{4-[3-methyl-4- | B | 502.0 | 5.00 |
| | (pyridin-3-yloxy)-phenylamino]- | | | |
| | quinazolin-6-yl}-prop-2-ynyl)- | | | |
| | acetamide | | | |
| 79 | N-(3-{4-[3-Chloro-4-(pyridin-3-yloxy)- | B | 522.0 | 5.28 |
| | phenylamino]-quinazolin-6-yl}-prop-2- | | | |
| | ynyl)-2-methanesulfonyl-acetamide | | | |
| 80 | (3-{4-[3-Methyl-4-(pyridin-3-yloxy)- | B | 456.2 | 6.02 |
| | phenylamino]-quinazolin-6-yl}-prop-2- | | | |
| | ynyl)-thiocarbamic acid S-methyl ester | | | |
| 81 | (3-{4-[3-Chloro-4-(pyridin-3-yloxy)- | B | 476.1 | 6.29 |
| | phenylamino]-quinazolin-6-yl}-prop-2- | | | |
| | ynyl)-thiocarbamic acid S-methyl ester | | | |
| 82 | [4-(2-Methyl-pyridin-3-yloxy)-phenyl]- | A | 436.1 | 4.24 |
| | (6-piperidin-4-ylethynyl-quinazolin-4- | | | |
| | yl)-amine | | | |
| 83 | (±)-[4-(2-Methyl-pyridin-3-yloxy)- | A | 436.0 | 4.85 |
| | phenyl]-(6-piperidin-3-ylethynyl- | | | |
| | quinazolin-4-yl)-amine | | | |
| 84 | N-(3-{4-[4-(2-Methyl-pyridin-3-yloxy)- | B | 424.1 | 4.85 |
| | phenylamino]-quinazolin-6-yl}-prop-2- | | | |
| | ynyl)-acetamide | | | |
| 85 | N-(3-{4-[3-Methyl-4-(pyridin-3-yloxy)- | B | 452.1 | 5.64 |
| | phenylamino]-quinazolin-yl}-prop-2- | | | |
| | ynyl)-2-oxo-propionamide | | | |
| 86 | N-(3-{4-[3-Chloro-4-(pyridin-3-yloxy)- | B | 474.3, | 5.93 |
| | phenylamino]-quinazolin-6-yl}-prop-2- | | 472.3 | |
| | ynyl)-2-oxo-propionamide | | | |
| 87 | N-(3-{4-[3-Methyl-4-(pyridin-3-yloxy)- | B | 496.2 | 5.56 |
| | phenylamino]-quinazolin-6-yl}-prop-2- | | | |
| | ynyl)-malonamic acid ethyl ester | | | |
| 88 | N-(3-{4-[3-Chloro-4-(pyridin-3-yloxy)- | B | 516.0 | 5.84 |
| | phenylamino]-quinazolin-6yl}-prop-2- | | | |
| | ynyl)-malonamic acid ethyl ester | | | |
| 89 | N-(1,1-Dimethyl-3-{4-[3-methyl-4- | B | 506.0 | 6.76 |
| | (pyridin-3-yloxy)-phenylamino]- | | | |
| | quinazolin-6-yl}-prop-2-ynyl)-2,2,2- | | | |
| | trifluoro-acetamide | | | |
| 90 | (±)-N-(1-Hydroxymethyl-3-{4-[3-methyl- | B | 454.1 | 4.47 |
| | 4-(pyridin-3-yloxy)-phenylamino]- | | | |
| | quinazolin-6-yl}-prop-2-ynyl)- | | | |
| | acetamide | | | |
| 99 | (±)-[3-Ethynyl-4-(pyridin-3-yloxy)- | A | 446.1 | 4.33 |
| | phenyl]-(6-piperidin-3-ylethynyl- | | | |
| | quinazolin-4-yl)-amine | | | |
| 100 | [3-Ethynyl-4-(pyridin-3-yloxy)-phenyl]- | A | 446.1 | 4.27 |
| | (6-piperidin-4-ylethynyl-quinazolin-4- | | | |
| | yl)-amine | | | |
| 101 | 3-{4-[3-Chloro-4-(pyridin-3-yloxy)- | B | 403.1 | 5.43 |
| | phenylamino]-quinazolin-6-yl}-prop-2- | | | |
| | yn-1-ol | | | |
| 102 | (±)-N-(1-Hydroxymethyl-3-{4-[3-methyl- | B | 468.1 | 4.66 |
| | 4-(6-methyl-pyridin-3-yloxy)- | | | |
| | phenylamino]-quinazolin-6-yl}-prop-2- | | | |
| | ynyl)-acetamide | | | |
| 103 | (±)-N-(3-{4-[3-Chloro-4-(pyridin-3- | B | 474.0 | 4.78 |
| | yloxy)-phenylamino]-quinazolin-6-yl}-1- | | | |
| | hydroxymethyl-prop-2-ynyl)-acetamide | | | |
| 104 | 2,2-Difluoro-N-(3-{4-[3-methyl-4-(6- | B | 474.2 | 5.83 |
| | methyl-pyridin-3-yloxy)-phenylamino]- | | | |
| | quinazolin-6-yl}-prop-2-ynyl)- | | | |
| | acetamide | | | |
| 105 | 2-Methanesulfonyl-N-(3-{4-[3-methyl-4- | B | 516.0 | 5.20 |
| | (6-methyl-pyridin-3-yloxy)- | | | |
| | phenylamino]-quinazolin-6-yl}-prop-2- | | | |
| | ynyl)-acetamide | | | |
| 106 | 2-Fluoro-N-(3-{4-[3-methyl-4-(pyridin- | B | 441.8 | 5.27 |
| | 3-yloxy)-phenylamino]-quinazolin-6-yl}- | | | |
| | prop-2-ynyl)-acetamide | | | |
| 107 | N-(3-{4-[3-Chloro-4-(pyridin-3-yloxy)- | B | 461.9 | 5.55 |
| | phenylamino]-quinazolin-6-yl}-prop-2- | | | |
| | ynyl)-2-fluoro-acetamide | | | |
| 108 | 2-Fluoro-N-(3-{4-[3-methyl-4-(6- | B | 456.2 | 5.47 |
| | methyl-pyridin-3-yloxy)-phenylamino]- | | | |
| | quinazolin-6-yl}-prop-2-ynyl)- | | | |
| | acetamide | | | |
| 109 | N-(3-{4-[3-Ethynyl-4-(pyridin-3-yloxy)- | B | 464.1 | 5.16 |
| | phenylamino]-quinazolin-6-yl}-prop-2- | | | |
| | ynyl)-2-methoxy-acetamide | | | |
| 110 | 2-Methoxy-N-(3-{4-[4-(2-methyl- | B | 454.2 | 5.15 |
| | pyridin-3-yloxy)-phenylamino]- | | | |
| | quinazolin-6-yl}-prop-2-ynyl)- | | | |
| | acetamide | | | |
| 111 | [4-(2-Chloro-pyridin-3-yloxy)-phenyl]- | A | 456.1 | 4.64 |
| | (6-piperidin-4-ylethynyl-quinazolin-4- | | | |
| | yl)-amine | | | |
| 112 | (±)-[4-(2-Chloro-pyridin-3-yloxy)- | A | 456.0 | 4.67 |
| | phenyl]-(6-piperidin-3-ylethynyl- | | | |
| | quinazolin-4-yl)-amine | | | |
| 113 | N-(3-{4-[4-(2-Chloro-pyridin-3-yloxy)- | B | 474.0 | 5.54 |
| | phenylamino]-quinazolin-6-yl}-prop-2- | | | |
| | ynyl)-2-methoxy-acetamide | | | |
| 114 | N-(3-{4-[4-(2-Chloro-pyridin-3-yloxy)- | B | 444.0 | 5.25 |
| | phenylamino]-quinazolin-6-yl}-prop-2- | | | |
| | ynyl)-acetamide | | | |
| 115 | N-(3-{4-[3-Ethynyl-4-(pyridin-3-yloxy)- | B | 434.1 | 4.88 |
| | phenylamino]-quinazolin-6-yl}-prop-2- | | | |
| | ynyl)-acetamide | | | |
| 116 | 1-(2-Chloro-ethyl)-3-(3-{4-[3-methyl-4- | D | 487.2 | 5.46 |
| | (pyridin-3-yloxy)-phenylamino]- | | | |
| | quinazolin-6-yl}-prop-2-ynyl)-urea | | | |
| 117 | (±)-[3-Fluoro-4-(2-methyl-pyridin-3- | A | 454.1 | 4.57 |
| | yloxy)-phenyl]-(6-piperidin-3-ylethynyl- | | | |
| | quinazolin-4-yl)-amine | | | |
| 118 | [3-Fluoro-4-(2-methyl-pyridin-3-yloxy)- | A | 454.1 | 4.56 |
| | phenyl]-(6-piperidin-4-ylethynyl- | | | |
| | quinazolin-4-yl)-amine | | | |
| 119 | N-(3-{4-[3-Fluoro-4-(2-methyl-pyridin- | B | 442.1 | 5.19 |
| | 3-yloxy)-phenylamino]-quinazolin-6-yl}- | | | |
| | prop-2-ynyl)-acetamide | | | |
| 120 | N-(3-{4-[3-Chloro-4-(6-methyl-pyridin- | B | 458.0 | 5.48 |
| | 3-yloxy)-phenylamino]-quinazolin-6-yl}- | | | |
| | prop-2-ynyl)-acetamide | | | |
| 121 | [3-Chloro-4-(6-methyl-pyridin-3-yloxy)- | A | 470.0 | 4.78 |
| | phenyl]-(6-piperidin-4-ylethynyl- | | | |
| | quinazolin-4-yl)-amine | | | |
| 122 | (±)-[3-Chloro-4-(6-methyl-pyridin-3- | A | 470.0 | 4.80 |
| | yloxy)-phenyl]-(6-piperidin-3-ylethynyl- | | | |
| | quinazolin-4-yl)-amine | | | |
| 123 | Acetic acid 3-{4-[3-methyl-4-(pyridin-3- | B | 425.1 | 6.34 |
| | yloxy)-phenylamino]-quinazolin-6-yl}- | | | |
| | prop-2-ynyl ester | | | |
| 124 | 3-{4-[3-Methyl-4-(6-methyl-pyridin-3- | B | 397.2 | 5.31 |
| | yloxy)-phenylamino]-quinazolin-6-yl}- | | | |
| | prop-2-yn-1-ol | | | |
| 125 | Acetic acid 3-{4-[3-methyl-4-(6-methyl- | B | 439.1 | 6.57 |
| | pyridin-3-yloxy)-phenylamino]- | | | |
| | quinazolin-6-yl}-prop-2-ynyl ester | | | |
| 126 | Acetic acid 3-{4-[3-chloro-4-(pyridin-3- | B | 445.1 | 6.66 |
| | yloxy)-phenylamino]-quinazolin-6-yl}- | | | |
| | prop-2-ynyl ester | | | |
| 127 | 2-Hydroxy-N-(3-{4-[3-methyl-4-(6- | A | 454.2 | 4.81 |
| | methyl-pyridin-3-yloxy)-phenylamino]- | | | |
| | quinazolin-6-yl}-prop-2-ynyl)- | | | |
| | acetamide | | | |
| 130 | N-(3-{4-[3-Methyl-4-(6-methyl-pyridin- | B | 438.0 | 5.20 |
| | 3-yloxy)-phenylamino]-quinazolin-6-yl}- | | | |
| | prop-2-ynyl)-acetamide | | | |
| 131 | R-Pyrrolidine-2-carboxylic acid (3-{4- | A | 493.0 | 4.42 |
| | [3-methyl-4-(6-methyl-pyridin-3-yloxy)- | | | |
| | phenylamino]-quinazolin-6-yl}-prop-2- | | | |
| | ynyl)-amide | | | |
| 132 | 2-(2-Hydroxy-ethylsulfanyl)-N-(3-{4-[3- | C | 513.9 | 5.07 |
| | methyl-4-(6-methyl-pyridin-3-yloxy)- | | | |
| | phenylamino]-quinazolin-6-yl}-prop-2- | | | |
| | ynyl)-acetamide | | | |
| 133 | (±)-2-Methanesulfinyl-N-(3-{4-[3- | B | 499.9 | 4.71 |
| | methyl-4-(6-methyl-pyridin-3-yloxy)- | | | |
| | phenylamino]-quinazolin-6-yl}-prop-2- | | | |
| | ynyl)-acetamide | | | |
| 134 | (3-{4-[3-Methyl-4-(6-methyl-pyridin-3- | B | 469.9 | 6.25 |
| | yloxy)-phenylamino]-quinazolin-6-yl}- | | | |
| | prop-2-ynyl)-thiocarbamic acid S- | | | |
| | methyl ester | | | |
| 135 | (±)-Tetrahydro-furan-3-carboxylic acid | B | 494.0 | 5.31 |
| | (3-{4-[3-methyl-4-(6-methyl-pyridin-3- | | | |
| | yloxy)-phenylamino]-quinazolin-6-yl}- | | | |
| | prop-2-ynyl)-amide | | | |
| 136 | N-(3-{4-[3-Methyl-4-(6-methyl-pyridin- | B | 465.9 | 5.87 |
| | 3-yloxy)-phenylamino]-quinazolin-6-yl}- | | | |
| | prop-2-ynyl)-2-oxo-propionamide | | | |
| 137 | N-(3-{4-[3-Methyl-4-(6-methyl-pyridin- | B | 510.0 | 5.77 |
| | 3-yloxy)-phenylamino]-quinazolin-6-yl}- | | | |
| | prop-2-ynyl)-malonamic acid ethyl | | | |
| | ester | | | |
| 138 | (6-Piperidin-4-ylethynyl-quinazolin-4- | A | 422.2 | 3.48 |
| | yl)-[4-(pyridin-2-yloxy)-phenyl]-amine | | | |
| 139 | (±)-(6-Piperidin-3-ylethynyl-quinazolin- | A | 422.2 | 3.51 |
| | 4-yl)-[4-(pyridin-2-yloxy)-phenyl]-amine | | | |
| 140 | N-(3-{4-[4-(Pyridin-2-yloxy)- | B | 410.1 | 3.81 |
| | phenylamino]-quinazolin-6-yl}-prop-2- | | | |
| | ynyl)-acetamide | | | |
| 141 | 2-Methylamino-N-(3-{4-[3-methyl-4-(6- | C | 467.0 | 4.26 |
| | methyl-pyridin-3-yloxy)-phenylamino]- | | | |
| | quinazolin-6-yl}-prop-2-ynyl)- | | | |
| | acetamide | | | |
| 142 | 2-Dimethylamino-N-(3-{4-[3-methyl-4- | C | 481.0 | 4.26 |
| | (6-methyl-pyridin-3-yloxy)- | | | |
| | phenylamino]-quinazolin-6-yl}-prop-2- | | | |
| | ynyl)-acetamide | | | |
| 143 | (±)-N-(3-{4-[3-Chloro-4-(6-methyl- | B | 488.0 | 4.99 |
| | pyridin-3-yloxy)-phenylamino]- | | | |
| | quinazolin-6-yl}-1-hydroxymethyl-prop- | | | |
| | 2-ynyl)-acetamide | | | |
| 144 | N-(3-{4-[3-Chloro-4-(6-methyl-pyridin- | C | 501.0 | 4.83 |
| | 3-yloxy)-phenylamino]-quinazolin-6-yl}- | | | |
| | prop-2-ynyl)-2-dimethylamino- | | | |
| | acetamide | | | |
| 145 | N-(3-{4-[3-Chloro-4-(6-methyl-pyridin- | B | 488.0 | 5.79 |
| | 3-yloxy)-phenylamino]-quinazolin-6-yl}- | | | |
| | prop-2-ynyl)-2-methoxy-acetamide | | | |
| 146 | N-(3-{4-[3-Chloro-4-(6-methyl-pyridin- | B | 476.0 | 5.79 |
| | 3-yloxy)-phenylamino]-quinazolin-6-yl}- | | | |
| | prop-2-ynyl)-2-fluoro-acetamide | | | |
| 147 | N-(3-{4-[3-Chloro-4-(6-methyl-pyridin- | B | 494.0 | 6.14 |
| | 3-yloxy)-phenylamino]-quinazolin-6-yl}- | | | |
| | prop-2-ynyl)-2,2-difluoro-acetamide | | | |
| 148 | E-3-{4-[3-Chloro-4-(pyridin-3-yloxy)- | E | 405.1 | 5.04 |
| | phenylamino]-quinazolin-6-yl}-prop-2- | | | |
| | en-1-ol | | | |
| 149 | 2-Methoxy-N-(3-{4-[4-(pyridin-2-yloxy)- | B | 440.8 | 4.05 |
| | phenylamino]-quinazolin-6-yl}-prop-2- | | | |
| | ynyl)-acetamide | | | |
| 150 | 1-Ethyl-3-(3-{4-[3-methyl-4-(6-methyl- | D | 467.2 | 5.36 |
| | pyridin-3-yloxy)-phenylamino]- | | | |
| | quinazolin-6-yl}-prop-2-ynyl)-urea | | | |
| 151 | 1-(3-{4-[3-Chloro-4-(pyridin-3-yloxy)- | D | 473.2 | 5.45 |
| | phenylamino]-quinazolin-6-yl}-prop-2- | | | |
| | ynyl)-3-ethyl-urea | | | |
| 152 | 1-Ethyl-3-(3-{4-[3-methyl-4-(pyridin-3- | D | 453.1 | 5.16 |
| | yloxy)-phenylamino]-quinazolin-6-yl}- | | | |
| | prop-2-ynyl)-urea | | | |
| 153 | 1-(3-{4-[3-Chloro-4-(6-methyl-pyridin-3- | D | 487.1 | 5.60 |
| | yloxy)-phenylamino]-quinazolin-6-yl}- | | | |
| | prop-2-ynyl)-3-ethyl-urea | | | |
| 154 | (±)-2-Hydroxy-N-(3-{4-[3-methyl-4- | A | 454.1 | 4.79 |
| | (pyridin-3-yloxy)-phenylamino]- | | | |
| | quinazolin-6-yl}-prop-2-ynyl)- | | | |
| | propionamide | | | |
| 155 | N-(3-{4-[3-Methyl-4-(2-methyl-pyridin- | B | 466.1 | 5.85 |
| | 3-yloxy)-phenylamino]-quinazolin-6-yl}- | | | |
| | prop-2-ynyl)-2-oxo-propionamide | | | |
| 156 | N-(3-{4-[3-Methyl-4-(2-methyl-pyridin- | B | 438.1 | 5.18 |
| | 3-yloxy)-phenylamino]-quinazolin-6-yl}- | | | |
| | prop-2-ynyl)-acetamide | | | |
| 157 | (±)-2-Hydroxy-N-(3-{4-[3-methyl-4-(6- | A | 468.0 | 4.98 |
| | methyl-pyridin-3-yloxy)-phenylamino]- | | | |
| | quinazolin-6-yl}-prop-2-ynyl)- | | | |
| | propionamide | | | |
| 158 | (±)-N-(3-{4-[3-Chloro-4-(6-methyl- | A | 488.0 | 5.32 |
| | pyridin-3-yloxy)-phenylamino]- | | | |
| | quinazolin-6-yl}-prop-2-ynyl)-2- | | | |
| | hydroxy-propionamide | | | |
| 159 | N-(3-{4-[3-Methyl-4-(6-methyl-pyridin- | C | 536.2 | 4.46 |
| | 3-yloxy)-phenylamino]-quinazolin-6-yl}- | | | |
| | prop-2-ynyl)-2-(4-methyl-piperazin-1- | | | |
| | yl)-acetamide | | | |
| 160 | 2-[Bis-(2-methoxy-ethyl)-amino]-N-(3- | C | 569.1 | 5.93 |
| | {4-[3-methyl-4-(6-methyl-pyridin-3- | | | |
| | yloxy)-phenylamino]-quinazolin-6-yl}- | | | |
| | prop-2-ynyl)-acetamide | | | |
| 161 | 2-(2-Hydroxy-ethylamino)-N-(3-{4-[3- | C | 483.0 | 4.11 |
| | methyl-4-(pyridin-3-yloxy)- | | | |
| | phenylamino]-quinazolin-6-yl}-prop-2- | | | |
| | ynyl)-acetamide | | | |
| 162 | N-(3-{4-[3-Chloro-4-(pyridin-3-yloxy)- | C | 487.0 | 4.65 |
| | phenylamino]-quinazolin-6-yl}-prop-2- | | | |
| | ynyl)-2-dimethylamino-acetamide | | | |
| 163 | N-(3-{4-[3-Chloro-4-(pyridin-3-yloxy)- | C | 473.0 | 4.42 |
| | phenylamino]-quinazolin-6-yl}-prop-2- | | | |
| | ynyl)-2-methylamino-acetamide | | | |
| 164 | N-(3-{4-[3-Chloro-4-(6-methyl-pyridin- | C | 487.1 | 4.60 |
| | 3-yloxy)-phenylamino]-quinazolin-6-yl}- | | | |
| | prop-2-ynyl)-2-methylamino-acetamide | | | |
| 165 | N-(3-{4-[3-Chloro-4-(6-methyl-pyridin- | A | 474.0 | 5.13 |
| | 3-yloxy)-phenylamino]-quinazolin-6-yl}- | | | |
| | prop-2-ynyl)-2-hydroxy-acetamide | | | |
| 166 | 1-(3-{4-[3-Chloro-4-(6-methyl-pyridin-3- | D | 501.8 | 5.98 |
| | yloxy)-phenylamino]-quinazolin-6-yl}- | | | |
| | prop-2-ynyl)-3-isopropyl-urea | | | |
| 167 | 1-Isopropyl-3-(3-{4-[3-methyl-4-(6- | D | 481.0 | 5.69 |
| | methyl-pyridin-3-yloxy)-phenylamino]- | | | |
| | quinazolin-6-yl}-prop-2-ynyl)-urea | | | |
| 168 | Morpholine-4-carboxylic acid (3-{4-[3- | D | 509.1 | 5.27 |
| | methyl-4-(6-methyl-pyridin-3-yloxy)- | | | |
| | phenylamino]-quinazolin-6-yl}-prop-2- | | | |
| | ynyl)-amide | | | |
| 169 | N-(3-{4-[3-Chloro-4-(6-methyl-pyridin- | C | 543.3 | 5.64 |
| | 3-yloxy)-phenylamino]-quinazolin-6-yl}- | | | |
| | prop-2-ynyl)-2-morpholin-4-yl- | | | |
| | acetamide | | | |
| 170 | N-(3-{4-[3-Methyl-4-(6-methyl-pyridin- | C | 522.8 | 5.37 |
| | 3-yloxy)-phenylamino]-quinazolin-6-yl}- | | | |
| | prop-2-ynyl)-2-morpholin-4-yl- | | | |
| | acetamide | | | |
| 171 | [6-(3-Amino-prop-1-ynyl)-quinazolin-4- | A | 396.3 | 4.05 |
| | yl]-[3-methyl-4-(6-methyl-pyridin-3- | | | |
| | yloxy)-phenyl]-amine | | | |
| 172 | E-[6-(3-Amino-propenyl)-quinazolin-4- | G | 398.2 | 3.87 |
| | yl]-[3-methyl-4-(6-methyl-pyridin-3- | | | |
| | yloxy)-phenyl]-amine | | | |
| 173 | E-[6-(3-Amino-propenyl)-quinazolin-4- | G | 418.0 | 4.26 |
| | yl]-[3-chloro-4-(6-methyl-pyridin-3- | | | |
| | yloxy)-phenyl]-amine | | | |
| 174 | 2-Hydroxy-N-(3-{4-[3-methyl-4-(pyridin- | A | 468.1 | 5.04 |
| | 3-yloxy)-phenylamino]-quinazolin-6-yl}- | | | |
| | prop-2-ynyl)-isobutyramide | | | |
| 175 | 2-Hydroxy-N-(3-{4-[3-methyl-4-(6- | A | 482.1 | 5.24 |
| | methyl-pyridin-3-yloxy)-phenylamino]- | | | |
| | quinazolin-6-yl}-prop-2-ynyl)- | | | |
| | isobutyramide | | | |
| 176 | N-(3-{4-[3-Chloro-4-(6-methyl-pyridin- | A | 502.0, | 5.55 |
| | 3-yloxy)-phenylamino]-quinazolin-6-yl}- | | 504.0 | |
| | prop-2-ynyl)-2-hydroxy-isobutyramide | | | |
| 177 | [6-(3-Amino-prop-1-ynyl)-quinazolin-4- | A | 416.2 | 4.25 |
| | yl]-[3-chloro-4-(6-methyl-pyridin-3- | | | |
| | yloxy)-phenyl]-amine | | | |
| 178 | N-(3-{4-[3-Methyl-4-(6-methyl-pyridin- | C | 535.3 | 5.99 |
| | 3-yloxy)-phenylamino]-quinazolin-6-yl}- | | | |
| | prop-2-ynyl)-2-(2,2,2-trifluoro- | | | |
| | ethylamino)-acetamide | | | |
| 179 | 1,1-Dimethyl-3-(3-{4-[3-methyl-4-(6- | D | 467.3 | 5.36 |
| | methyl-pyridin-3-yloxy)-phenylamino]- | | | |
| | quinazolin-6-yl}-prop-2-ynyl)-urea | | | |
| 180 | 3-(3-{4-[3-Chloro-4-(6-methyl-pyridin-3- | D | 515.0 | 6.32 |
| | yloxy)-phenylamino]-quinazolin-6-yl}- | | | |
| | prop-2-ynyl)-1,1-diethyl-urea | | | |
| 181 | 3-(3-{4-[3-Chloro-4-(6-methyl-pyridin-3- | D | 487.1 | 5.70 |
| | yloxy)-phenylamino]-quinazolin-6-yl}- | | | |
| | prop-2-ynyl)-1,1-dimethyl-urea | | | |
| 182 | E-N-(3-{4-[3-Methyl-4-(6-methyl- | G | 440.3 | 4.74 |
| | pyridin-3-yloxy)-phenylamino]- | | | |
| | quinazolin-6-yl}-allyl)-acetamide | | | |
| 183 | E-2-Methoxy-N-(3-{4-[3-methyl-4-(6- | G | 470.1 | 5.05 |
| | methyl-pyridin-3-yloxy)-phenylamino]- | | | |
| | quinazolin-6-yl}-allyl)-acetamide | | | |
| 184 | Morpholine-4-carboxylic acid (3-{4-[3- | D | 529.0 | 5.58 |
| | chloro-4-(6-methyl-pyridin-3-yloxy)- | | | |
| | phenylamino]-quinazolin-6-yl}-prop-2- | | | |
| | ynyl)-amide | | | |
| 185 | Pyrrolidine-1-carboxylic acid (3-{4-[3- | D | 513.0 | 6.00 |
| | chloro-4-(6-methyl-pyridin-3-yloxy)- | | | |
| | phenylamino]-quinazolin-6-yl}-prop-2- | | | |
| | ynyl)-amide | | | |
| 186 | 1-(3-{4-[3-Chloro-4-(6-methyl-pyridin-3- | D | 473.0 | 5.37 |
| | yloxy)-phenylamino]-quinazolin-6-yl}- | | | |
| | prop-2-ynyl)-3-methyl-urea | | | |
| 187 | 1-(3-{4-[3-Chloro-4-(6-methyl-pyridin-3- | D | 501.0 | 6.03 |
| | yloxy)-phenylamino]-quinazolin-6-yl}- | | | |
| | prop-2-ynyl)-3-propyl-urea | | | |
| 188 | 1-tert-Butyl-3-(3-{4-[3-chloro-4-(6- | D | 515.0 | 6.56 |
| | methyl-pyridin-3-yloxy)-phenylamino]- | | | |
| | quinazolin-6-yl}-prop-2-ynyl)-urea | | | |
| 189 | 2S-Hydroxy-N-(3-{4-[3-methyl-4-(6- | B | 468.0 | 4.95 |
| | methyl-pyridin-3-yloxy)-phenylamino]- | | | |
| | quinazolin-6-yl}-prop-2-ynyl)- | | | |
| | propionamide | | | |
| 190 | 1-(3-{4-[3-Chloro-4-(6-methyl-pyridin-3- | D | 540.7 | 6.19 |
| | yloxy)-phenylamino]-quinazolin-6-yl}- | | | |
| | prop-2-ynyl)-3-(2,2,2-trifluoro-ethyl)- | | | |
| | urea | | | |
| 191 | (±)-Azetidine-2-carboxylic acid (3-{4-[3- | A | 465.2 | 4.21 |
| | methyl-4-(pyridin-3-yloxy)- | | | |
| | phenylamino]-quinazolin-6-yl}-prop-2- | | | |
| | ynyl)-amide | | | |
| 192 | (±)-Azetidine-2-carboxylic acid (3-{4-[3- | A | 479.3 | 4.41 |
| | methyl-4-(6-methyl-pyridin-3-yloxy)- | | | |
| | phenylamino]-quinazolin-6-yl}-prop-2- | | | |
| | ynyl)-amide | | | |
| 193 | (±)-Azetidine-2-carboxylic acid (3-{4-[3- | A | 499.3 | 4.70 |
| | chloro-4-(6-methyl-pyridin-3-yloxy)- | | | |
| | phenylamino]-quinazolin-6-yl}-prop-2- | | | |
| | ynyl)-amide | | | |
| 194 | 1-Hydroxy-cyclopropanecarboxylic | B | 480.0 | 5.20 |
| | acid (3-{4-[3-methyl-4-(6-methyl- | | | |
| | pyridin-3-yloxy)-phenylamino]- | | | |
| | quinazolin-6-yl}-prop-2-ynyl)-amide | | | |
| 194 | N-(3-{4-[3-Methyl-4-(6-methyl-pyridin- | B | 452.1 | 5.55 |
| | 3-yloxy)-phenylamino]-quinazolin-6-yl}- | | | |
| | prop-2-ynyl)-propionamide | | | |
| 195 | N-(3-{4-[3-Methyl-4-(6-methyl-pyridin- | B | 466.1 | 5.88 |
| | 3-yloxy)-phenylamino]-quinazolin-6-yl}- | | | |
| | prop-2-ynyl)-butyramide | | | |
| 196 | N-(3-{4-[3-Methyl-4-(6-methyl-pyridin- | B | 466.1 | 5.88 |
| | 3-yloxy)-phenylamino]-quinazolin-6-yl}- | | | |
| | prop-2-ynyl)-isobutyramide | | | |
| 197 | 2-Ethoxy-N-(3-{4-[3-methyl-4-(6- | B | 482.1 | 5.89 |
| | methyl-pyridin-3-yloxy)-phenylamino]- | | | |
| | quinazolin-6-yl}-prop-2-ynyl)- | | | |
| | acetamide | | | |
| 198 | N-(3-{4-[3-Methyl-4-(6-methyl-pyridin- | B | 484.0 | 5.76 |
| | 3-yloxy)-phenylamino]-quinazolin-6-yl}- | | | |
| | prop-2-ynyl)-2-methylsulfanyl- | | | |
| | acetamide | | | |
| 199 | Cyclopropanecarboxylic acid (3-{4-[3- | B | 464.1 | 5.76 |
| | methyl-4-(6-methyl-pyridin-3-yloxy)- | | | |
| | phenylamino]-quinazolin-6-yl}-prop-2- | | | |
| | ynyl)-amide | | | |
| 200 | Cyclobutanecarboxylic acid (3-{4-[3- | B | 478.1 | 6.07 |
| | methyl-4-(6-methyl-pyridin-3-yloxy)- | | | |
| | phenylamino]-quinazolin-6-yl}-prop-2- | | | |
| | ynyl)-amide | | | |
| 201 | [6-(3-Amino-prop-1-ynyl)-quinazolin-4- | A | 382.1 | 4.02 |
| | yl]-[3-methyl-4-(pyridin-3-yloxy)- | | | |
| | phenyl]-amine | | | |
| 202 | Isoxazole-5-carboxylic acid (3-{4-[3- | B | 491.0 | 5.78 |
| | methyl-4-(6-methyl-pyridin-3-yloxy)- | | | |
| | phenylamino]-quinazolin-6-yl}-prop-2- | | | |
| | ynyl)-amide | | | |
| 203 | N-Methyl-N-(3-{4-[3-methyl-4-(6- | B | 452.5 | 5.79 |
| | methyl-pyridin-3-yloxy)-phenylamino]- | | | |
| | quinazolin-6-yl}-prop-2-ynyl)- | | | |
| | acetamide | | | |
| 204 | 2-Methoxy-N-(1-methyl-3-{4-[3-methyl- | B | 481.9 | 5.86 |
| | 4-(6-methyl-pyridin-3-yloxy)- | | | |
| | phenylamino]-quinazolin-6-yl}-prop-2- | | | |
| | ynyl)-acetamide | | | |
| 205 | N-(1,1-Dimethyl-3-{4-[3-methyl-4-(6- | B | 466.2 | 5.82 |
| | methyl-pyridin-3-yloxy)-phenylamino]- | | | |
| | quinazolin-6-yl}-prop-2-ynyl)- | | | |
| | acetamide | | | |
| 206 | 2R-Hydroxy-N-(3-{4-[3-methyl-4-(6- | B | 468.0 | 4.95 |
| | methyl-pyridin-3-yloxy)-phenylamino]- | | | |
| | quinazolin-6-yl}-prop-2-ynyl)- | | | |
| | propionamide | | | |
| 207 | E-Cyclopropanecarboxylic acid (3-{4- | G | 466.1 | 5.41 |
| | [3-methyl-4-(6-methyl-pyridin-3-yloxy)- | | | |
| | phenylamino]-quinazolin-6-yl}-allyl)- | | | |
| | amide | | | |
| 208 | E-N-(3-{4-[3-Methyl-4-(6-methyl- | G | 454.1 | 5.07 |
| | pyridin-3-yloxy)-phenylamino]- | | | |
| | quinazolin-6-yl}-allyl)-propionamide | | | |
| 209 | E-2-Ethoxy-N-(3-{4-[3-methyl-4-(6- | G | 484.0 | 5.54 |
| | methyl-pyridin-3-yloxy)-phenylamino]- | | | |
| | quinazolin-6-yl}-allyl)-acetamide | | | |
| 210 | E-(±)-2-Methoxy-N-(3-{4-[3-methyl-4- | G | 484.1 | 5.45 |
| | (6-methyl-pyridin-3-yloxy)- | | | |
| | phenylamino]-quinazolin-6-yl}-allyl)- | | | |
| | propionamide | | | |
| 211 | E-2-Fluoro-N-(3-{4-[3-methyl-4-(6- | G | 458.1 | 5.48 |
| | methyl-pyridin-3-yloxy)-phenylamino]- | | | |
| | quinazolin-6-yl}-allyl)-acetamide | | | |
| 212 | 2-Methoxy-N-(1-{4-[3-methyl-4-(6- | B | 508.0 | 6.17 |
| | methyl-pyridin-3-yloxy)-phenylamino]- | | | |
| | quinazolin-6-ylethynyl}-cyclobutyl)- | | | |
| | acetamide | | | |
| 213 | N-(1-{4-[3-Methyl-4-(6-methyl-pyridin- | B | 478.0 | 5.90 |
| | 3-yloxy)-phenylamino]-quinazolin-6- | | | |
| | ylethynyl}-cyclobutyl)-acetamide | | | |
| 214 | E-N-(3-{4-[3-Chloro-4-(6-methyl- | G | 478.0 | 5.55 |
| | pyridin-3-yloxy)-phenylamino]- | | | |
| | quinazolin-6-yl}-allyl)-2-fluoro- | | | |
| | acetamide | | | |
| 215 | E-Cyclopropanecarboxylic acid (3-{4- | G | 485.7 | 5.77 |
| | [3-chloro-4-(6-methyl-pyridin-3-yloxy)- | | | |
| | phenylamino]-quinazolin-6-yl}-allyl)- | | | |
| | amide | | | |
| 216 | [6-(1-Amino-cyclobutylethynyl)- | A | 436.0 | 4.87 |
| | quinazolin-4-yl]-[3-methyl-4-(6-methyl- | | | |
| | pyridin-3-yloxy)-phenyl]-amine | | | |
| 217 | (±)-2-Methoxymethyl-pyrrolidine-1- | D | 537.1 | 6.13 |
| | carboxylic acid (3-{4-[3-methyl-4-(6- | | | |
| | methyl-pyridin-3-yloxy)-phenylamino]- | | | |
| | quinazolin-6-yl}-prop-2-ynyl)-amide | | | |
| 218 | Piperazine-1-carboxylic acid (3-{4-[3- | D | 508.1 | 4.28 |
| | methyl-4-(6-methyl-pyridin-3-yloxy)- | | | |
| | phenylamino]-quinazolin-6-yl}-prop-2- | | | |
| | ynyl)-amide | | | |
| 219 | 3-(3-{4-[3-Chloro-4-(6-methyl-pyridin-3- | D | 531.0 | 5.41 |
| | yloxy)-phenylamino]-quinazolin-6-yl}- | | | |
| | prop-2-ynyl)-1-ethyl-1-(2-hydroxy- | | | |
| | ethyl)-urea | | | |
| 220 | [6-(1-Amino-cyclopropylethynyl)- | A | 422.1 | 5.11 |
| | quinazolin-4-yl]-[3-methyl-4-(6-methyl- | | | |
| | pyridin-3-yloxy)-phenyl]-amine | | | |
| 221 | E-3-{4-[3-Methyl-4-(6-methyl-pyridin-3- | E | 399.2 | 4.93 |
| | yloxy)-phenylamino]-quinazolin-6-yl}- | | | |
| | prop-2-en-1-ol | | | |
| 222 | N-(3-{4-[3-Chloro-4-(6-methyl-pyridin- | B | 532.0 | 5.86 |
| | 3-yloxy)-phenylamino]-quinazolin-6-yl}- | | | |
| | prop-2-ynyl)-2-(2-methoxy-ethoxy)- | | | |
| | acetamide | | | |
| 223 | N-(3-{4-[3-Chloro-4-(6-methyl-pyridin- | B | 486.0 | 6.17 |
| | 3-yloxy)-phenylamino]-quinazolin-6-yl}- | | | |
| | prop-2-ynyl)-2-oxo-propionamide | | | |
| 224 | N-(3-{4-[3-Chloro-4-(6-methyl-pyridin- | C | 534.0 | 5.57 |
| | 3-yloxy)-phenylamino]-quinazolin-6-yl}- | | | |
| | prop-2-ynyl)-2-(2-hydroxy- | | | |
| | ethylsulfanyl)-acetamide | | | |
| 225 | N-(3-{4-[3-Chloro-4-(6-methyl-pyridin- | B | 504.0 | 6.04 |
| | 3-yloxy)-phenylamino]-quinazolin-6-yl}- | | | |
| | prop-2-ynyl)-2-methylsulfanyl- | | | |
| | acetamide | | | |
| 226 | Pyrrolidine-2R-carboxylic acid (3-{4-[3- | A | 513.4 | 4.86 |
| | chloro-4-(6-methyl-pyridin-3-yloxy)- | | | |
| | phenylamino]-quinazolin-6-yl}-prop-2- | | | |
| | ynyl)-amide | | | |
| 227 | Pyrrolidine-2R-carboxylic acid (3-{4-[3- | A | 499.0 | 4.45 |
| | chloro-4-(pyridin-3-yloxy)- | | | |
| | phenylamino]-quinazolin-6-yl}-prop-2- | | | |
| | ynyl)-amide | | | |
| 228 | (±)-2-Methanesulfinyl-N-(3-{4-[3- | B | 486.1 | 4.52 |
| | methyl-4-(pyridin-3-yloxy)- | | | |
| | phenylamino]-quinazolin-6-yl}-prop-2- | | | |
| | ynyl)-acetamide | | | |
| 229 | (±)-2-Methanesulfinyl-N-(3-{4-[3- | B | 506.0 | 4.81 |
| | chloro-4-(pyridin-3-yloxy)- | | | |
| | phenylamino]-quinazolin-6-yl}-prop-2- | | | |
| | ynyl)-acetamide | | | |
| 230 | (±)-Tetrahydro-furan-3-carboxylic acid | B | 480.1 | 5.11 |
| | (3-{4-[3-methyl-4-(pyridin-3-yloxy)- | | | |
| | phenylamino]-quinazolin-6-yl}-prop-2- | | | |
| | ynyl)-amide | | | |
| 231 | 2-Hydroxy-N-(3-{4-[3-methyl-4-(pyridin- | A | 440.3 | 4.60 |
| | 3-yloxy)-phenylamino]-quinazolin-6-yl}- | | | |
| | prop-2-ynyl)-acetamide | | | |
| 232 | 2-Ethoxy-N-(3-{4-[3-methyl-4-(pyridin- | B | 467.9 | 5.62 |
| | 3-yloxy)-phenylamino]-quinazolin-6-yl}- | | | |
| | prop-2-ynyl)-acetamide | | | |
| 233 | [3-Methyl-4-(pyridin-3-yloxy)-phenyl]- | A | 436.6 | 4.35 |
| | (6-piperidin-4-ylethynyl-quinazolin-4- | | | |
| | yl)-amine | | | |
| 234 | Cyclobutanecarboxylic acid (3-{4-[3- | B | 464.0 | 5.78 |
| | methyl-4-(pyridin-3-yloxy)- | | | |
| | phenylamino]-quinazolin-6-yl}-prop-2- | | | |
| | ynyl)-amide | | | |
| 235 | Cyclopropanecarboxylic acid (3-{4-[3- | B | 450.0 | 5.44 |
| | methyl-4-(pyridin-3-yloxy)- | | | |
| | phenylamino]-quinazolin-6-yl}-prop-2- | | | |
| | ynyl)-amide | | | |
| 236 | [3-Methyl-4-(pyridin-2-yloxy)-phenyl]- | A | 436.0 | 4.64 |
| | (6-piperidin-3-ylethynyl-quinazolin-4- | | | |
| | yl)-amine | | | |
| 237 | (6-Azetidin-3-ylethynyl-quinazolin-4-yl)- | A | 407.9 | 4.10 |
| | [3-methyl-4-(pyridin-3-yloxy)-phenyl]- | | | |
| | amine | | | |
| 238 | N-(1,1-Dimethyl-3-{4-[3-methyl-4- | B | 481.9 | 5.96 |
| | (pyridin-3-yloxy)-phenylamino]- | | | |
| | quinazolin-6-yl}-prop-2-ynyl)-2- | | | |
| | methoxy-acetamide | | | |
| 239 | 2-[4-(3-{4-[3-Methyl-4-(pyridin-3-yloxy)- | A | 495.4 | 4.10 |
| | phenylamino]-quinazolin-6-yl}-prop-2- | | | |
| | ynyl)-piperazin-1-yl]-ethanol | | | |
| 240 | (±)-2-Methoxy-N-(1-methyl-3-{4-[3- | B | 467.9 | 5.57 |
| | methyl-4-(pyridin-3-yloxy)- | | | |
| | phenylamino]-quinazolin-6-yl}-prop-2- | | | |
| | ynyl)-acetamide | | | |
| 241 | [3-Methyl-4-(pyridin-3-yloxy)-phenyl]- | A | 436.0 | 4.48 |
| | (6-piperidin-3R-ylethynyl-quinazolin-4- | | | |
| | yl)-amine | | | |
| 242 | [3-Methyl-4-(pyridin-3-yloxy)-phenyl]- | A | 436.0 | 4.48 |
| | (6-piperidin-3S-ylethynyl-quinazolin-4- | | | |
| | yl)-amine | | | |
| 243 | (±)-[3-Methyl-4-(pyridin-3-yloxy)- | A | 422.0 | 4.30 |
| | phenyl]-(6-pyrrolidin-3-ylethynyl- | | | |
| | quinazolin-4-yl)-amine | | | |

TABLE 2

| Example No. | Name | Method | LRMS | HPLC RT |
|---|---|---|---|---|
| 244 | 1-(2-Methoxy-ethyl)-1-methyl-3-(3-{4- | D | 511.1 | 5.61 |
| | [3-methyl-4-(6-methyl-pyridin-3-yloxy)- | | | |
| | phenylamino]-quinazolin-6-yl}-prop-2- | | | |
| | ynyl)-urea | | | |
| 245 | (±)-2-Hydroxymethyl-pyrrolidine-1- | D | 523.1 | 5.19 |
| | carboxylic acid (3-{4-[3-methyl-4-(6- | | | |
| | methyl-pyridin-3-yloxy)-phenylamino]- | | | |
| | quinazolin-6-yl}-prop-2-ynyl)-amide | | | |
| 246 | (±)-3-Hydroxy-pyrrolidine-1-carboxylic | D | 509.1 | 4.75 |
| | acid (3-{4-[3-methyl-4-(6-methyl- | | | |
| | pyridin-3-yloxy)-phenylamino]- | | | |
| | quinazolin-6-yl}-prop-2-ynyl)-amide | | | |
| 247 | Cis- and trans-2,5-Dimethyl- | D | 521.1 | 6.38, |
| | pyrrolidine-1-carboxylic acid (3-{4-[3- | | | 6.28 |
| | methyl-4-(6-methyl-pyridin-3-yloxy)- | | | |
| | phenylamino]-quinazolin-6-yl}-prop-2- | | | |
| | ynyl)-amide | | | |
| 248 | 1-Isobutyl-1-methyl-3-(3-{4-[3-methyl- | D | 509.1 | 6.45 |
| | 4-(6-methyl-pyridin-3-yloxy)- | | | |
| | phenylamino]-quinazolin-6-yl}-prop-2- | | | |
| | ynyl)-urea | | | |
| 249 | N-(1-{4-[3-Methyl-4-(6-methyl-pyridin- | B | 464.0 | 5.46 |
| | 3-yloxy)-phenylamino]-quinazolin-6- | | | |
| | ylethynyl}-cyclopropyl)-acetamide | | | |
| 250 | 2-Methoxy-N-(1-{4-[3-methyl-4-(6- | B | 5.76 | 493.7 |
| | methyl-pyridin-3-yloxy)-phenylamino]- | | | |
| | quinazolin-6-ylethynyl}-cyclopropyl)- | | | |
| | acetamide | | | |
| 251 | E-N-(3-{4-[3-Chloro-4-(6-methyl- | G | 474.0 | 5.53 |
| | pyridin-3-yloxy)-phenylamino]- | | | |
| | quinazolin-6-yl}-allyl)-propionamide | | | |
| 252 | E-N-(3-{4-[3-Chloro-4-(6-methyl- | G | 504.0 | 5.67 |
| | pyridin-3-yloxy)-phenylamino]- | | | |
| | quinazolin-6-yl}-allyl)-2-methoxy- | | | |
| | propionamide | | | |
| 253 | E-N-(3-{4-[3-Chloro-4-(6-methyl- | G | 489.7 | 5.52 |
| | pyridin-3-yloxy)-phenylamino]- | | | |
| | quinazolin-6-yl}-allyl)-2-methoxy- | | | |
| | acetamide | | | |
| 254 | E-N-(3-{4-[3-Chloro-4-(6-methyl- | G | 504.0 | 5.89 |
| | pyridin-3-yloxy)-phenylamino]- | | | |
| | quinazolin-6-yl}-allyl)-2-ethoxy- | | | |
| | acetamide | | | |
| 255 | (3-{4-[3-Methyl-4-(6-methyl-pyridin-3- | B | 496.3 | 7.11 |
| | yloxy)-phenylamino]-quinazolin-6-yl}- | | | |
| | prop-2-ynyl)-carbamic acid tert-butyl | | | |
| | ester | | | |
| 256 | 2-(R)-Hydroxy-N-(3-{4-[3-methyl-4-(6- | B | 468.0 | 5.04 |
| | methyl-pyridin-3-yloxy)-phenylamino]- | | | |
| | quinazolin-6-yl}-prop-2-ynyl)- | | | |
| | propionamide | | | |
| 257 | Cyclobutanecarboxylic acid (3-{4-[3- | B | 498.0, | 6.36 |
| | chloro-4-(6-methyl-pyridin-3-yloxy)- | | 500.0 | |
| | phenylamino]-quinazolin-6-yl}-prop-2- | | | |
| | ynyl)-amide | | | |
| 258 | N-(3-{4-[3-Chloro-4-(6-methyl-pyridin- | B | 472.0, | 5.86 |
| | 3-yloxy)-phenylamino]-quinazolin-6-yl}- | | 474.0 | |
| | prop-2-ynyl)-propionamide | | | |
| 259 | Cyclopropanecarboxylic acid (3-{4-[3- | B | 484.0, | 6.06 |
| | chloro-4-(6-methyl-pyridin-3-yloxy)- | | 486.0 | |
| | phenylamino]-quinazolin-6-yl}-prop-2- | | | |
| | ynyl)-amide | | | |
| 260 | N-(3-{4-[3-Chloro-4-(6-methyl-pyridin- | B | 486.1, | 6.17 |
| | 3-yloxy)-phenylamino]-quinazolin-6-yl}- | | 488.1 | |
| | prop-2-ynyl)-isobutyramide | | | |
| 261 | (±)-N-(3-{4-[3-Chloro-4-(6-methyl- | B | 502.0, | 6.00 |
| | pyridin-3-yloxy)-phenylamino]- | | 504.0 | |
| | quinazolin-6-yl}-prop-2-ynyl)-2- | | | |
| | methoxy-propionamide | | | |
| 262 | (±)-2-Methoxy-N-(3-{4-[3-methyl-4-(6- | B | 482.1 | 5.73 |
| | methyl-pyridin-3-yloxy)-phenylamino]- | | | |
| | quinazolin-6-yl}-prop-2-ynyl)- | | | |
| | propionamide | | | |
| 263 | 5-Oxo-pyrrolidine-2R-carboxylic acid | B | 507.1 | 4.73 |
| | (3-{4-[3-methyl-4-(6-methyl-pyridin-3- | | | |
| | yloxy)-phenylamino]-quinazolin-6-yl}- | | | |
| | prop-2-ynyl)- | | | |
| | amide | | | |
| 264 | E-1-Hydroxy-cyclopropanecarboxylic | I | 482.0 | 4.65 |
| | acid (3-{4-[3-methyl-4-(6-methyl- | | | |
| | pyridin-3-yloxy)-phenylamino]- | | | |
| | quinazolin-6-yl}-allyl)-amide | | | |
| 265 | E-2S-Hydroxy-N-(3-{4-[3-methyl-4-(6- | I | 470.1 | 4.56 |
| | methyl-pyridin-3-yloxy)-phenylamino]- | | | |
| | quinazolin-6-yl}-allyl)-propionamide | | | |
| 266 | E-2R-Hydroxy-N-(3-{4-[3-methyl-4-(6- | I | 470.1 | 4.60 |
| | methyl-pyridin-3-yloxy)-phenylamino]- | | | |
| | quinazolin-6-yl}-allyl)-propionamide | | | |
| 267 | E-2-Hydroxy-N-(3-{4-[3-methyl-4-(6- | I | 456.1 | 4.51 |
| | methyl-pyridin-3-yloxy)-phenylamino]- | | | |
| | quinazolin-6-yl}-allyl)-acetamide | | | |
| 268 | 1-Cyanomethyl-3-(3-{4-[3-methyl-4-(6- | D | 478.1 | 5.26 |
| | methyl-pyridin-3-yloxy)-phenylamino]- | | | |
| | quinazolin-6-yl}-prop-2-ynyl)-urea | | | |
| 269 | 1-Cyclobutyl-3-(3-{4-[3-methyl-4-(6- | D | 492.8 | 5.90 |
| | methyl-pyridin-3-yloxy)-phenylamino]- | | | |
| | quinazolin-6-yl}-prop-2-ynyl)-urea | | | |
| 270 | 1,1,3-Trimethyl-3-(3-{4-[3-methyl-4-(6- | D | 481.1 | 6.30 |
| | methyl-pyridin-3-yloxy)-phenylamino]- | | | |
| | quinazolin-6-yl}-prop-2-ynyl)-urea | | | |
| 271 | 1-(3-{4-[3-Chloro-4-(6-methyl-pyridin-3- | D | 501.1 | 6.52 |
| | yloxy)-phenylamino]-quinazolin-6-yl}- | | | |
| | prop-2-ynyl)-1,3,3-trimethyl-urea | | | |
| 272 | 1-Ethyl-1-(2-hydroxy-ethyl)-3-(3-{4-[3- | D | 511.1 | 5.20 |
| | methyl-4-(6-methyl-pyridin-3-yloxy)- | | | |
| | phenylamino]-quinazolin-6-yl}-prop-2- | | | |
| | ynyl)-urea | | | |
| 273 | (±)-3-Dimethylamino-pyrrolidine-1- | D | 536.1 | 4.40 |
| | carboxylic acid (3-{4-[3-methyl-4-(6- | | | |
| | methyl-pyridin-3-yloxy)-phenylamino]- | | | |
| | quinazolin-6-yl}-prop-2-ynyl)-amide | | | |
| 274 | Morpholine-4-carboxylic acid (1- | D | 523.4 | 5.58 |
| | methyl-3-{4-[3-methyl-4-(6-methyl- | | | |
| | pyridin-3-yloxy)-phenylamino]- | | | |
| | quinazolin-6-yl}-prop-2-ynyl)-amide | | | |
| 275 | Exo-6-Amino-3-aza- | D | 520.9 | 4.28 |
| | bicyclo[3.1.0]hexane-3-carboxylic acid | | | |
| | (3-{4-[3-methyl-4-(6-methyl-pyridin-3- | | | |
| | yloxy)-phenylamino]-quinazolin-6-yl}- | | | |
| | prop-2-ynyl)-amide | | | |
| 276 | Exo-6-Hydroxymethyl-3-aza- | D | 535.1 | 4.98 |
| | bicyclo[3.1.0]hexane-3-carboxylic acid | | | |
| | (3-{4-[3-methyl-4-(6-methyl-pyridin-3- | | | |
| | yloxy)-phenylamino]-quinazolin-6-yl}- | | | |
| | prop-2-ynyl)-amide | | | |
| 277 | (3-{4-[3-Methyl-4-(6-methyl-pyridin-3- | D | 439.8 | 4.81 |
| | yloxy)-phenylamino]-quinazolin-6-yl}- | | | |
| | prop-2-ynyl)-urea | | | |
| 278 | E-N-(3-{4-[3-Chloro-4-(6-methyl- | I | 476.0 | 4.86 |
| | pyridin-3-yloxy)-phenylamino]- | | | |
| | quinazolin-6-yl}-allyl)-2-hydroxy- | | | |
| | acetamide | | | |
| 279 | Piperazine-1-carboxylic acid (1,1- | D | 536.1 | 4.74 |
| | dimethyl-3-{4-[3-methyl-4-(6-methyl- | | | |
| | pyridin-3-yloxy)-phenylamino]- | | | |
| | quinazolin-6-yl}-prop-2-ynyl)-amide | | | |
| 280 | 1-(1,1-Dimethyl-3-{4-[3-methyl-4-(6- | D | 495.3 | 6.11 |
| | methyl-pyridin-3-yloxy)-phenylamino]- | | | |
| | quinazolin-6-yl}-prop-2-ynyl)-3-ethyl- | | | |
| | urea | | | |
| 281 | Morpholine-4-carboxylic acid (1,1- | D | 537.3 | 6.02 |
| | dimethyl-3-{4-[3-methyl-4-(6-methyl- | | | |
| | pyridin-3-yloxy)-phenylamino]- | | | |
| | quinazolin-6-yl}-prop-2-ynyl)-amide | | | |
| 282 | 1,3-Dimethyl-1-(3-{4-[3-methyl-4-(6- | D | 467.1 | 5.51 |
| | methyl-pyridin-3-yloxy)-phenylamino]- | | | |
| | quinazolin-6-yl}-prop-2-ynyl)-urea | | | |
| 283 | N-(3-{4-[3-Chloro-4-(6-methyl-pyridin- | B | 5.02, | 5.74 |
| | 3-yloxy)-phenylamino]-quinazolin-6-yl}- | | 5.04 | |
| | 1,1-dimethyl-prop-2-ynyl)-2-hydroxy- | | | |
| | acetamide | | | |
| 284 | N-(1,1-Dimethyl-3-{4-[3-methyl-4-(6- | B | 482.2 | 5.46 |
| | methyl-pyridin-3-yloxy)-phenylamino]- | | | |
| | quinazolin-6-yl}-prop-2-ynyl)-2- | | | |
| | hydroxy-acetamide | | | |
| 285 | E-1,1-Diethyl-3-(3-{4-[3-methyl-4-(6- | H | 497.1 | 5.72 |
| | methyl-pyridin-3-yloxy)-phenylamino]- | | | |
| | quinazolin-6-yl}-allyl)-urea | | | |
| 286 | E-Pyrrolidine-1-carboxylic acid (3-{4- | H | 495.1 | 5.40 |
| | [3-methyl-4-(6-methyl-pyridin-3-yloxy)- | | | |
| | phenylamino]-quinazolin-6-yl}-allyl)- | | | |
| | amide | | | |
| 287 | E-1-Ethyl-3-(3-{4-[3-methyl-4-(6- | H | 469.1 | 4.80 |
| | methyl-pyridin-3-yloxy)-phenylamino]- | | | |
| | quinazolin-6-yl}-allyl)-urea | | | |
| 288 | E-Morpholine-4-carboxylic acid (3-{4- | H | 511.1 | 4.75 |
| | [3-methyl-4-(6-methyl-pyridin-3-yloxy)- | | | |
| | phenylamino]-quinazolin-6-yl}-allyl)- | | | |
| | amide | | | |
| 289 | (±)-1-Ethyl-1-(2-hydroxy-ethyl)-3-(1- | D | 525.1 | 5.51 |
| | methyl-3-{4-[3-methyl-4-(6-methyl- | | | |
| | pyridin-3-yloxy)-phenylamino]- | | | |
| | quinazolin-6-yl}-prop-2-ynyl)-urea | | | |
| 290 | (±)-1-Ethyl-3-(1-methyl-3-{4-[3-methyl- | D | 481.1 | 5.68 |
| | 4-(6-methyl-pyridin-3-yloxy)- | | | |
| | phenylamino]-quinazolin-6-yl}-prop-2- | | | |
| | ynyl)-urea | | | |
| 291 | 4-Methyl-piperazine-1-carboxylic acid | D | 522.3 | 4.44 |
| | (3-{4-[3-methyl-4-(6-methyl-pyridin-3- | | | |
| | yloxy)-phenylamino]-quinazolin-6-yl}- | | | |
| | prop-2-ynyl)-amide | | | |
| 292 | N-(3-{4-[3-Chloro-4-(6-methyl-pyridin- | B | 483.1 | 5.73 |
| | 3-yloxy)-phenylamino]-quinazolin-6-yl}- | | | |
| | prop-2-ynyl)-2-cyano-acetamide | | | |
| 293 | 2-Cyano-N-(3-{4-[3-methyl-4-(6- | B | 463.1 | 5.44 |
| | methyl-pyridin-3-yloxy)-phenylamino]- | | | |
| | quinazolin-6-yl}-prop-2-ynyl)- | | | |
| | acetamide | | | |
| 294 | E-N-(3-{4-[3-Methyl-4-(6-methyl- | G | 486.3 | 5.33 |
| | pyridin-3-yloxy)-phenylamino]- | | | |
| | quinazolin-6-yl}-allyl)-2-methylsulfanyl- | | | |
| | acetamide | | | |
| 295 | E-5-Oxo-tetrahydro-furan-2R- | G | 510.2 | 5.58 |
| | carboxylic acid (3-{4-[3-methyl-4-(6- | | | |
| | methyl-pyridin-3-yloxy)-phenylamino]- | | | |
| | quinazolin-6-yl}-allyl)-amide | | | |
| 296 | E-N-(3-{4-[3-Methyl-4-(6-methyl- | G | 476.0 | 5.36 |
| | pyridin-3-yloxy)-phenylamino]- | | | |
| | quinazolin-6-yl}-allyl)- | | | |
| | methanesulfonamide | | | |
| 297 | (±)-5-{4-[3-Methyl-4-(6-methyl-pyridin- | B | 466.1 | 5.22 |
| | 3-yloxy)-phenylamino]-quinazolin-6- | | | |
| | ylethynyl}-morpholin-3-one | | | |
| 298 | E-N-(3-{4-[3-Chloro-4-(6-methyl- | I | 490.1 | 5.06 |
| | pyridin-3-yloxy)-phenylamino]- | | | |
| | quinazolin-6-yl}-allyl)-2S-hydroxy- | | | |
| | propionamide | | | |
| 299 | E-1-Hydroxy-cyclopropanecarboxylic | I | 502.2 | 5.24 |
| | acid (3-{4-[3-chloro-4-(6-methyl- | | | |
| | pyridin-3-yloxy)-phenylamino]- | | | |
| | quinazolin-6-yl}-allyl)-amide | | | |
| 300 | E-N-(3-{4-[3-Chloro-4-(6-methyl- | I | 504.2 | 5.24 |
| | pyridin-3-yloxy)-phenylamino]- | | | |
| | quinazolin-6-yl}-allyl)-2-hydroxy- | | | |
| | isobutyramide | | | |
| 301 | (±)-E-N-(3-{4-[3-Chloro-4-(6-methyl- | I | 490.0 | 5.07 |
| | pyridin-3-yloxy)-phenylamino]- | | | |
| | quinazolin-6-yl}-allyl)-2-hydroxy- | | | |
| | propionamide | | | |
| 302 | 2R-Amino-N-(3-{4-[3-chloro-4-(6- | A | 487.1 | 4.54 |
| | methyl-pyridin-3-yloxy)-phenylamino]- | | | |
| | quinazolin-6-yl}-prop-2-ynyl)- | | | |
| | propionamide | | | |
| 303 | 2R-Amino-N-(3-{4-[3-methyl-4-(6- | A | 467.2 | 4.35 |
| | methyl-pyridin-3-yloxy)-phenylamino]- | | | |
| | quinazolin-6-yl}-prop-2-ynyl)- | | | |
| | propionamide | | | |
| 304 | (±)-4-{4-[3-Methyl-4-(6-methyl-pyridin- | | 452.2 | 5.40 |
| | 3-yloxy)-phenylamino]-quinazolin-6- | | | |
| | ylethynyl}-oxazolidin-2-one | | | |
| 305 | (±)-E-3,3,3-Trifluoro-2-hydroxy-N-(3-{4- | I | 524.1 | 5.52 |
| | [3-methyl-4-(6-methyl-pyridin-3-yloxy)- | | | |
| | phenylamino]-quinazolin-6-yl}-allyl)- | | | |
| | propionamide | | | |
| 306 | (±)-E-2-Hydroxy-3-methyl-N-(3-{4-[3- | I | 498.2 | 5.49 |
| | methyl-4-(6-methyl-pyridin-3-yloxy)- | | | |
| | phenylamino]-quinazolin-6-yl}-allyl)- | | | |
| | butyramide | | | |
| 307 | (±)-2-Methoxymethyl-pyrrolidine-1- | D | 557.1 | 6.42 |
| | carboxylic acid (3-{4-[3-chloro-4-(6- | | | |
| | methyl-pyridin-3-yloxy)-phenylamino]- | | | |
| | quinazolin-6-yl}-prop-2-ynyl)-amide | | | |
| 308 | (±)-2-Hydroxymethyl-pyrrolidine-1- | D | 543.2 | 5.61 |
| | carboxylic acid (3-{4-[3-chloro-4-(6- | | | |
| | methyl-pyridin-3-yloxy)-phenylamino]- | | | |
| | quinazolin-6-yl}-prop-2-ynyl)-amide | | | |
| 309 | (±)-1-(3-{4-[3-Chloro-4-(6-methyl- | D | 529.2 | 6.87 |
| | pyridin-3-yloxy)-phenylamino]- | | | |
| | quinazolin-6-yl}-prop-2-ynyl)-3-(1,2- | | | |
| | dimethyl-propyl)-urea | | | |
| 310 | (±)-1-(3-{4-[3-Chloro-4-(6-methyl- | D | 529.2 | 6.89 |
| | pyridin-3-yloxy)-phenylamino]- | | | |
| | quinazolin-6-yl}-prop-2-ynyl)-3-(1,1- | | | |
| | dimethyl-propyl)-urea | | | |
| 311 | (±)-1-(3-{4-[3-Chloro-4-(6-methyl- | D | 531.1 | 5.41 |
| | pyridin-3-yloxy)-phenylamino]- | | | |
| | quinazolin-6-yl}-prop-2-ynyl)-3-(1- | | | |
| | hydroxymethyl-propyl)-urea | | | |
| 312 | 1-(3-{4-[3-Chloro-4-(6-methyl-pyridin-3- | D | 529.1 | 6.63 |
| | yloxy)-phenylamino]-quinazolin-6-yl}- | | | |
| | prop-2-ynyl)-3-(1-ethyl-propyl)-urea | | | |
| 313 | (±)-1-sec-Butyl-3-(3-{4-[3-chloro-4-(6- | D | 515.1 | 6.32 |
| | methyl-pyridin-3-yloxy)-phenylamino]- | | | |
| | quinazolin-6-yl}-prop-2-ynyl)-urea | | | |
| 314 | (±)-1-(1,1-Dimethyl-propyl)-3-(3-{4-[3- | D | 509.2 | 6.60 |
| | methyl-4-(6-methyl-pyridin-3-yloxy)- | | | |
| | phenylamino]-quinazolin-6-yl}-prop-2- | | | |
| | ynyl)-urea | | | |
| 315 | (±)-1-(1-Hydroxymethyl-propyl)-3-(3-{4- | D | 511.2 | 5.13 |
| | [3-methyl-4-(6-methyl-pyridin-3-yloxy)- | | | |
| | phenylamino]-quinazolin-6-yl}-prop-2- | | | |
| | ynyl)-urea | | | |
| 316 | 1-(1-Ethyl-propyl)-3-(3-{4-[3-methyl-4- | D | 509.3 | 6.35 |
| | (6-methyl-pyridin-3-yloxy)- | | | |
| | phenylamino]-quinazolin-6-yl}-prop-2- | | | |
| | ynyl)-urea | | | |
| 317 | (±)-1-sec-Butyl-3-(3-{4-[3-methyl-4-(6- | D | 495.3 | 6.07 |
| | methyl-pyridin-3-yloxy)-phenylamino]- | | | |
| | quinazolin-6-yl}-prop-2-ynyl)-urea | | | |
| 318 | Azetidine-1-carboxylic acid (3-{4-[3- | D | 479.2 | 5.46 |
| | methyl-4-(6-methyl-pyridin-3-yloxy)- | | | |
| | phenylamino]-quinazolin-6-yl}-prop-2- | | | |
| | ynyl)-amide | | | |
| 319 | 1-(1,2-Dimethyl-propyl)-3-(3-{4-[3- | D | 509.2 | 6.36 |
| | methyl-4-(6-methyl-pyridin-3-yloxy)- | | | |
| | phenylamino]-quinazolin-6-yl}-prop-2- | | | |
| | ynyl)-urea | | | |
| 320 | Piperidine-1-carboxylic acid (3-{4-[3- | D | 507.3 | 6.21 |
| | methyl-4-(6-methyl-pyridin-3-yloxy)- | | | |
| | phenylamino]-quinazolin-6-yl}-prop-2- | | | |
| | ynyl)-amide | | | |
| 321 | E-Pyridine-2-carboxylic acid (3-{4-[3- | G | 503.3 | 6.11 |
| | methyl-4-(6-methyl-pyridin-3-yloxy)- | | | |
| | phenylamino]-quinazolin-6-yl}-allyl)- | | | |
| | amide | | | |
| 322 | E-2-Isopropoxy-N-(3-{4-[3-methyl-4-(6- | G | 498.3 | 5.94 |
| | methyl-pyridin-3-yloxy)-phenylamino]- | | | |
| | quinazolin-6-yl}-allyl)-acetamide | | | |
| 323 | E-N-(3-{4-[3-Methyl-4-(6-methyl- | G | 538.1 | 6.51 |
| | pyridin-3-yloxy)-phenylamino]- | | | |
| | quinazolin-6-yl}-allyl)- | | | |
| | benzenesulfonamide | | | |
| 324 | E-Ethanesulfonic acid (3-{4-[3-methyl- | G | 490.3 | 5.62 |
| | 4-(6-methyl-pyridin-3-yloxy)- | | | |
| | phenylamino]-quinazolin-6-yl}-allyl)- | | | |
| | amide | | | |
| 325 | E-1H-Imidazole-4-carboxylic acid (3- | G | 492.3 | 5.53 |
| | {4-[3-methyl-4-(6-methyl-pyridin-3- | | | |
| | yloxy)-phenylamino]-quinazolin-6-yl}- | | | |
| | allyl)-amide | | | |
| 326 | E-Isoxazole-5-carboxylic acid (3-{4-[3- | G | 493.2 | 5.41 |
| | methyl-4-(6-methyl-pyridin-3-yloxy)- | | | |
| | phenylamino]-quinazolin-6-yl}-allyl)- | | | |
| | amide | | | |
| 327 | E-Pyrrolidine-1-carboxylic acid (3-{4- | H | 515.2 | 5.77 |
| | [3-chloro-4-(6-methyl-pyridin-3-yloxy)- | | | |
| | phenylamino]-quinazolin-6-yl}-allyl)- | | | |
| | amide | | | |
| 328 | E-Morpholine-4-carboxylic acid (3-{4- | H | 531.1 | 5.20 |
| | [3-chloro-4-(6-methyl-pyridin-3-yloxy)- | | | |
| | phenylamino]-quinazolin-6-yl}-allyl)- | | | |
| | amide | | | |
| 329 | E-N-(3-{4-[3-Chloro-4-(6-methyl- | G | 506.1 | 5.81 |
| | pyridin-3-yloxy)-phenylamino]- | | | |
| | quinazolin-6-yl}-allyl)-2-methylsulfanyl- | | | |
| | acetamide | | | |
| 330 | E-5-Oxo-tetrahydro-furan-2R- | G | 530.2 | 5.44 |
| | carboxylic acid (3-{4-[3-chloro-4-(6- | | | |
| | methyl-pyridin-3-yloxy)-phenylamino]- | | | |
| | quinazolin-6-yl}-allyl)-amide | | | |
| 331 | E-N-(3-{4-[3-Chloro-4-(6-methyl- | G | 530.2 | 6.34 |
| | pyridin-3-yloxy)-phenylamino]- | | | |
| | quinazolin-6-yl}-allyl)-2- | | | |
| | cyclopropylmethoxy-acetamide | | | |
| 332 | (±)-E-N-(3-{4-[3-Chloro-4-(6-methyl- | I | 518.2 | 5.73 |
| | pyridin-3-yloxy)-phenylamino]- | | | |
| | quinazolin-6-yl}-allyl)-2-hydroxy-3- | | | |
| | methyl-butyramide | | | |
| 333 | E-N-(3-{4-[3-Chloro-4-(6-methyl- | G | 496.1 | 5.72 |
| | pyridin-3-yloxy)-phenylamino]- | | | |
| | quinazolin-6-yl}-allyl)- | | | |
| | methanesulfonamide | | | |
| 334 | E-2R-Hydroxymethyl-pyrrolidine-1- | H | 525.2 | 4.91 |
| | carboxylic acid (3-{4-[3-methyl-4-(6- | | | |
| | methyl-pyridin-3-yloxy)-phenylamino]- | | | |
| | quinazolin-6-yl}-allyl)-amide | | | |
| 335 | E-2S-Hydroxymethyl-pyrrolidine-1- | H | 525.2 | 4.92 |
| | carboxylic acid (3-{4-[3-methyl-4-(6- | | | |
| | methyl-pyridin-3-yloxy)-phenylamino]- | | | |
| | quinazolin-6-yl}-allyl)-amide | | | |
| 336 | E-2-Cyclopropylmethoxy-N-(3-{4-[3- | G | 510.3 | 6.00 |
| | methyl-4-(6-methyl-pyridin-3-yloxy)- | | | |
| | phenylamino]-quinazolin-6-yl}-allyl)- | | | |
| | acetamide | | | |
| 337 | E-1-Isopropyl-3-(3-{4-[3-methyl-4-(6- | H | 483.2 | 5.33 |
| | methyl-pyridin-3-yloxy)-phenylamino]- | | | |
| | quinazolin-6-yl}-allyl)-urea | | | |
| 338 | Azetidine-1-carboxylic acid (3-{4-[3- | D | 499.2 | 5.73 |
| | chloro-4-(6-methyl-pyridin-3-yloxy)- | | | |
| | phenylamino]-quinazolin-6-yl}-prop-2- | | | |
| | ynyl)-amide | | | |
| 339 | Piperazine-1-carboxylic acid (3-{4-[3- | D | 528.2 | 4.65 |
| | chloro-4-(6-methyl-pyridin-3-yloxy)- | | | |
| | phenylamino]-quinazolin-6-yl}-prop-2- | | | |
| | ynyl)-amide | | | |
| 340 | 2-Methoxy-N-(3-{7-methoxy-4-[3- | B | 498.2 | 5.47 |
| | methyl-4-(6-methyl-pyridin-3-yloxy)- | | | |
| | phenylamino]-quinazolin-6-yl}-prop-2- | | | |
| | ynyl)-acetamide | | | |
| 341 | N-(3-{4-[3-Chloro-4-(6-methyl-pyridin- | B | 518.2 | 5.76 |
| | 3-yloxy)-phenylamino]-7-methoxy- | | | |
| | quinazolin-6-yl}-prop-2-ynyl)-2- | | | |
| | methoxy-acetamide | | | |
| 342 | 3-(3-{4-[3-Chloro-4-(6-methyl-pyridin-3- | D | 531.2 | 5.92 |
| | yloxy)-phenylamino]-quinazolin-6-yl}- | | | |
| | prop-2-ynyl)-1-(2-methoxy-ethyl)-1- | | | |
| | methyl-urea | | | |
| 343 | N-(3-{7-Methoxy-4-[3-methyl-4-(6- | B | 468.2 | 5.21 |
| | methyl-pyridin-3-yloxy)-phenylamino]- | | | |
| | quinazolin-6-yl}-prop-2-ynyl)- | | | |
| | acetamide | | | |
| 344 | N-(3-{4-[3-Chloro-4-(6-methyl-pyridin- | B | 488.2 | 5.50 |
| | 3-yloxy)-phenylamino]-7-methoxy- | | | |
| | quinazolin-6-yl}-prop-2-ynyl)- | | | |
| | acetamide | | | |
| 345 | 1,1-Diisopropyl-3-(3-{7-methoxy-4-[3- | D | 553.3 | 6.79 |
| | methyl-4-(6-methyl-pyridin-3-yloxy)- | | | |
| | phenylamino]-quinazolin-6-yl}-prop-2- | | | |
| | ynyl)-urea | | | |
| 346 | (±)-2-Methyl-piperidine-1-carboxylic | D | 521.3 | 6.53 |
| | acid (3-{4-[3-methyl-4-(6-methyl- | | | |
| | pyridin-3-yloxy)-phenylamino]- | | | |
| | quinazolin-6-yl}-prop-2-ynyl)-amide | | | |
| 347 | E-Azetidine-2S-carboxylic acid (3-{4- | G | 481.3 | 4.10 |
| | [3-methyl-4-(6-methyl-pyridin-3-yloxy)- | | | |
| | phenylamino]-quinazolin-6-yl}-allyl)- | | | |
| | amide | | | |
| 348 | E-1-Amino-cyclopropanecarboxylic | G | 481.3 | 4.40 |
| | acid (3-{4-[3-methyl-4-(6-methyl- | | | |
| | pyridin-3-yloxy)-phenylamino]- | | | |
| | quinazolin-6-yl}-allyl)-amide | | | |
| 349 | E-2-Amino-N-(3-{4-[3-methyl-4-(6- | G | 483.3 | 4.12 |
| | methyl-pyridin-3-yloxy)-phenylamino]- | | | |
| | quinazolin-6-yl}-allyl)-isobutyramide | | | |
| 350 | E-5-Oxo-pyrrolidine-2R-carboxylic acid | G | 509.2 | 4.45 |
| | (3-{4-[3-methyl-4-(6-methyl-pyridin-3- | | | |
| | yloxy)-phenylamino]-quinazolin-6-yl}- | | | |
| | allyl)-amide | | | |
| 351 | E-2R-Amino-N-(3-{4-[3-methyl-4-(6- | G | 469.3 | 4.09 |
| | methyl-pyridin-3-yloxy)-phenylamino]- | | | |
| | quinazolin-6-yl}-allyl)-propionamide | | | |
| 352 | E-2S-Amino-N-(3-{4-[3-methyl-4-(6- | G | 469.3 | 4.09 |
| | methyl-pyridin-3-yloxy)-phenylamino]- | | | |
| | quinazolin-6-yl}-allyl)-propionamide | | | |
| 353 | E-5-Oxo-pyrrolidine-2R-carboxylic acid | G | 509.2 | 4.42 |
| | (3-{4-[3-methyl-4-(6-methyl-pyridin-3- | | | |
| | yloxy)-phenylamino]-quinazolin-6-yl}- | | | |
| | allyl)-amide | | | |
| 354 | E-Isoxazole-5-carboxylic acid (3-{4-[3- | G | 513.0 | 5.86 |
| | chloro-4-(6-methyl-pyridin-3-yloxy)- | | | |
| | phenylamino]-quinazolin-6-yl}-allyl)- | | | |
| | amide | | | |
| 355 | E-3-(3-{4-[3-Chloro-4-(6-methyl- | H | 517.2 | 6.11 |
| | pyridin-3-yloxy)-phenylamino]- | | | |
| | quinazolin-6-yl}-allyl)-1,1-diethyl-urea | | | |
| 356 | E-Pyridine-2-carboxylic acid (3-{4-[3- | G | 523.1 | 6.47 |
| | chloro-4-(6-methyl-pyridin-3-yloxy)- | | | |
| | phenylamino]-quinazolin-6-yl}-allyl)- | | | |
| | amide | | | |
| 357 | N-(3-{4-[3-Methyl-4-(6-methyl-pyridin- | B | 474.2 | 5.66 |
| | 3-yloxy)-phenylamino]-quinazolin-6-yl}- | | | |
| | prop-2-ynyl)-methanesulfonamide | | | |
| 358 | N-(3-{4-[3-Chloro-4-(6-methyl-pyridin- | B | 494.1 | 5.93 |
| | 3-yloxy)-phenylamino]-quinazolin-6-yl}- | | | |
| | prop-2-ynyl)-methanesulfonamide | | | |

Utilizing methods A through I and the appropriate starting materials (prepared according to methodology known in the art), the following compounds, which are part of the present invention, may be prepared:

Z-2-Methoxy-N-(3-{4-[3-methyl-4-(6-methyl-pyridin-3-yloxy)-phenylamino]-quinazolin-6-yl}-allyl)-acetamide

E-2-(2-Fluoro-ethoxy)-N-(3-{4-[3-methyl-4-(6-methyl-pyridin-3-yloxy)-phenylamino]-quinazolin-6-yl}-allyl)-acetamide

Z-N-(3-{4-[3-Chloro-4-(6-methyl-pyridin-3-yloxy)-phenylamino]-quinazolin-6-yl}-allyl)-2-fluoro-acetamide

2-Hydroxy-N-(1-{4-[3-methyl-4-(6-methyl-pyridin-3-yloxy)-phenylamino]-quinazolin-6-ylethynyl}-cyclopropyl)-acetamide

E-2-Methoxy-N-(3-{4-[3-methyl-4-(6-methyl-pyridin-3-yloxy)-phenylamino]-quinazolin-6-yl}-allyl)-isobutyramide

1-Ethyl-3-(1-{4-[3-methyl-4-(6-methyl-pyridin-3-yloxy)-phenylamino]-quinazolin-6-ylethynyl}-cyclopropyl)-urea

1-Ethyl-3-[1-(2-{4-[3-methyl-4-(6-methyl-pyridin-3-yloxy)-phenylamino]-quinazolin-6-yl}-ethyl)-cyclopropyl]-urea

3-Methoxy-azetidine-1-carboxylic acid (3-{4-[3-methyl-4-(6-methyl-pyridin-3-yloxy)-phenylamino]-quinazolin-6-yl}-prop-2-ynyl)-amide

N-(3-{7-(2-Methoxy-ethoxy)-4-[3-methyl-4-(6-methyl-pyridin-3-yloxy)-phenylamino]-quinazolin-6-yl}-prop-2-ynyl)-acetamide

E-1-Methoxy-cyclopropanecarboxylic acid (3-{4-[3-methyl-4-(6-methyl-pyridin-3-yloxy)-phenylamino]-quinazolin-6-yl}-allyl)-amide

N-(3-{4-[3-Methyl-4-(2-methyl-pyrimidin-5-yloxy)-phenylamino]-quinazolin-6-yl}-prop-2-ynyl)-acetamide

(±)-E-1-(2-Fluoro-ethyl )-3-(1-methyl-3-{4-[3-methyl-4-(6-methyl-pyridin-3-yloxy)-phenylamino]-quinazolin-6-yl}-allyl)-urea

E-N-[1-(2-{4-[3-Chloro-4-(6-methyl-pyridin-3-yloxy)-phenylamino]-quinazolin-6-yl}-vinyl)-cyclopropyl]-methanesulfonamide

(±)-E-Tetrahydro-furan-3-carboxylic acid (3-{4-[3-chloro-4-(6-methyl-pyridin-3-yloxy)-phenylamino]-quinazolin-6-yl}-allyl)-amide

E-Morpholine-4-carboxylic acid (3-{4-[3-methyl-4-(6-methyl-pyridin-3-yloxy)-phenylamino]-quinazolin-6-yl}-allyl)-amide

N-[1-(2-{4-[3-Chloro-4-(6-methyl-pyridin-3-yloxy)-phenylamino]-quinazolin-6-yl}-ethyl)-cyclopropyl]-methanesulfonamide

(±)-E-Tetrahydro-furan-2-carboxylic acid (3-{4-[3-chloro-4-(6-methyl-pyridin-3-yloxy)-phenylamino]-quinazolin-6-yl}-allyl)-amide

(±)-Ethanesulfonic acid (1-methyl-3-{4-[3-methyl-4-(6-methyl-pyridin-3-yloxy)-phenylamino]-quinazolin-6-yl}-prop-2-ynyl)-amide

(±)-Pyridine-2-carboxylic acid (1-methyl-3-{4-[3-methyl-4-(6-methyl-pyridin-3-yloxy)-phenylamino]-quinazolin-6-yl}-prop-2-ynyl)-amide

and the pharmaceutically acceptable salts, solvates and prodrugs of the foregoing compounds.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. All patents, applications, publications, test methods, literature, and other materials cited herein are hereby incorporated herein by reference in their entireties.

1. A method of treating a mammal having a cancer, comprising: administering to said mammal in need of such treatment, sequentially in either order, simultaneously, or both, (i) a therapeutically effective amount of a compound of the formula 1 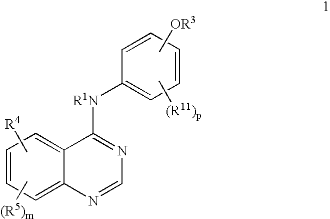
or a pharmaceutically acceptable salt, solvate or prodrug thereof, wherein:
m is an integer from 0 to 3;
p is an integer from 0 to 4;
each R¹ and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;
R⁴ is —(CR¹⁶R¹⁷)ₘ—C≡C—(CR¹⁶R¹⁷)ₜR⁹, —(CR¹⁶R¹⁷)ₘ—C═C—(CR¹⁶R¹⁷)ₜ—R⁹, —(CR¹⁶R¹⁷)ₘ—C≡C—(CR¹⁶R¹⁷)ₖR¹³, —(CR¹⁶R¹⁷)ₘ—C═C—(CR¹⁶R¹⁷)ₖR¹³, or —(CR¹⁶R¹⁷)ₜR⁹, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconnection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR⁶¹R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or —OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R¹⁵;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;
each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally substituted with a group selected from hydroxy, halo, C₁-C₄ alkyl, C₁-C₄ alkoxy and —NR¹R², and
(ii) an amount of an additional therapeutic agent.

2. The method according to claim 1, wherein the additional therapeutic agent is selected from the group consisting of an antitumor agent, alkylating agent, antimetabolite, antibiotic, plant-derived antitumor agent, camptothecin derivative, tyrosine kinase inhibitor, antibody, interferon, and biological response modifier.

3. The method according to claim 1, wherein the additional therapeutic agent is selected from the group consisting of a 5-fluoruracil, bevacizumab, bicalutamide, buserelin, carboplatin, cetuximab, cisplatin, CP-547,632, CP-751,871, ticilimumab, cyproterone acetate, diethylstilbestrol, dexamethasone, docetaxel, doxorubicin, epirubicin, erlotinib, estramustine, exemestane, flutamide, gefitinib, gemcitabine, goserelin, hydrocortisone, irinotecan, ixabepilone ketoconazole, letrozole, leuprolide, mitoxantrone, nilutamine, oxaliplatin, paclitaxel, pertuzumab, prednisone, PROMUNE™, SU-11248, tamoxifen, and vinorelbine and combinations thereof.

4. The method according to claims 1 or 2, wherein the compound of formula 1 is selected from the group consisting of:
(±)-[3-Methyl-4-(pyridin-3-yloxy)-phenyl]-(6-piperidin-3-ylethynyl-quinazolin-4-yl)-amine;
2-Methoxy-N-(3-{4-[3-methyl-4-(pyridin-3-yloxy)-phenylamino]-quinazolin-6-yl}-prop-2-ynyl)-acetamide
(±)-[3-Methyl-4-(6-methyl-pyridin-3-yloxy)-phenyl]-(6-piperidin-3-ylethynyl-quinazolin-4-yl)-amine;
[3-Methyl-4-(6-methyl-pyridin-3-yloxy)-phenyl]-(6-piperidin-4-ylethynyl-quinazolin-4-yl)-amine;
2-Methoxy-N-(3-{4-[3-methyl-4-(6-methyl-pyridin-3-yloxy)-phenylamino]-quinazolin-6-yl}-prop-2-ynyl)-acetamide;
2-Fluoro-N-(3-{4-[3-methyl-4-(6-methyl-pyridin-3-yloxy)-phenylamino]-quinazolin-6-yl}-prop-2-ynyl)-acetamide;
E-2-Methoxy-N-(3-{4-[3-methyl-4-(6-methyl-pyridin-3-yloxy)-phenylamino]-quinazolin-6-yl}-allyl)-acetamide;
[3-Methyl-4-(pyridin-3-yloxy)-phenyl]-(6-piperidin-4-ylethynyl-quinazolin-4-yl)-amine;
2-Methoxy-N-(1-{4-[3-methyl-4-(6-methyl-pyridin-3-yloxy)-phenylamino]-quinazolin-6-ylethynyl}-cyclopropyl)-acetamide;
E-N-(3-{4-[3-Chloro-4-(6-methyl-pyridin-3-yloxy)-phenylamino]-quinazolin-6-yl}-allyl)-2-methoxy-acetamide;
N-(3-{4-[3-Chloro-4-(6-methyl-pyridin-3-yloxy)-phenylamino]-quinazolin-6-yl}-prop-2-ynyl)-acetamide;
N-(3-{4-[3-Methyl-4-(6-methyl-pyridin-3-yloxy)-phenylamino]-quinazolin-6-yl}-prop-2-ynyl)-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
1-Ethyl-3-(3-{4-[3-methyl-4-(6-methyl-pyridin-3-yloxy)-phenylamino]-quinazolin-6-yl}-prop-2-ynyl)-urea;
Piperazine-1-carboxylic acid (3-{4-[3-methyl-4-(6-methyl-pyridin-3-yloxy)-phenylamino]-quinazolin-6-yl}-prop-2-ynyl)-amide;
(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;
2-Dimethylamino-N-(3-{4-[3-methyl-4-(pyridin-3-yloxy)-phenylamino]-quinazolin-6-yl}-prop-2-ynyl)-acetamide;
E-N-(3-{4-[3-Methyl-4-(6-methyl-pyridin-3-yloxy)-phenylamino]-quinazolin-6-yl}-allyl)-methanesulfonamide;
Isoxazole-5-carboxylic acid (3-{4-[3-methyl-4-(6-methyl-pyridin-3-yloxy)-phenylamino]-quinazolin-6-yl}-prop-2-ynyl)-amide;
1-(1,1-Dimethyl-3-{4-[3-methyl-4-(6-methyl-pyridin-3-yloxy)-phenylamino]-quinazolin-6-yl}-prop-2-ynyl)-3-ethyl-urea;
and the pharmaceutically acceptable salts, prodrugs and solvates of the foregoing compounds.

5. The method according to claim 4, wherein the compound of formula 1 is E-2-Methoxy-N-(3-{4-[3-methyl-4-(6-methyl-pyridin-3-yloxy)-phenylamino]-quinazolin-6-yl}-allyl)-acetamide.

6. The method according to claim 1, wherein the compound of formula 1 is administered orally, buccally, sublingually, vaginally, intraduodenally, parenterally, topically, or rectally.

7. The method according to claim 1, wherein the cancer is selected from the group consisting of: lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system, primary CNS lymphoma, spinal axis tumors, brain stem glioma, pituitary adenoma, or a combination of one or more of the foregoing cancers.

8. The method according to any one of the preceding claims claim 7, wherein the cancer is selected from the group consisting of ovarian cancer and breast cancer.

9. A composition comprising (i) a therapeutically effective amount of a compound of the formula 1 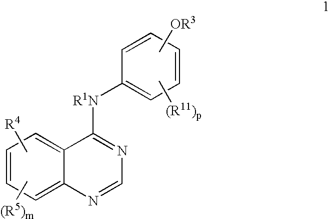
or a pharmaceutically acceptable salt, solvate or prodrug thereof, wherein:
m is an integer from 0 to 3;
p is an integer from 0 to 4;
each R¹ and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;
R⁴ is —(CR¹⁶R¹⁷)ₘ—C≡C—(CR¹⁶R¹⁷)ₜR⁹, —(CR¹⁶R¹⁷)ₘ—C═C—(CR¹⁶R¹⁷)ₜ—R⁹, —(CR¹⁶R¹⁷)ₘ—C≡C—(CR¹⁶R¹⁷)ₖR¹³, —(CR¹⁶R¹⁷)ₘ—C═C—(CR¹⁶R¹⁷)ₖR¹³, or —(CR¹⁶R¹⁷)ₜR⁹, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo (═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to a nitrogen atom (including the same nitrogen atom or two separate nitrogen atoms in proximity to each other through interconnection by, for instance, —C(O) or —SO₂—), can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo (═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo (═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms in which from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or —OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo (═O) moiety, and the aryl and heterocyclic moieties of the foregoing R¹⁵ groups are optionally substituted with 1 to 3 R⁸ substituents;
each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally substituted with a group selected from hydroxy, halo, C₁-C₄ alkyl, C₁-C₄ alkoxy and —NR¹R², and
(ii) an amount of an additional therapeutic agent.

10. The composition according to claim 9, wherein the additional therapeutic agent is selected from the group consisting of an antitumor agent, alkylating agent, antimetabolite, antibiotic, plant-derived antitumor agent, camptothecin derivative, tyrosine kinase inhibitor, antibody, interferon, and biological response modifier.

11. The composition according to claim 9, wherein the additional therapeutic agent is selected from the group consisting of a 5-fluoruracil, bevacizumab, bicalutamide, buserelin, carboplatin, cetuximab, cisplatin, CP-547,632, CP-751,871, ticilimumab, cyproterone acetate, diethylstilbestrol, dexamethasone, docetaxel, doxorubicin, epirubicin, erlotinib, estramustine, exemestane, flutamide, gefitinib, gemcitabine, goserelin, hydrocortisone, irinotecan, ixabepilone ketoconazole, letrozole, leuprolide, mitoxantrone, nilutamine, oxaliplatin, paclitaxel, pertuzumab, prednisone, PROMUNE™, SU-11248, tamoxifen, and vinorelbine and combinations thereof.

12. The composition according to claim 9, wherein the compound of formula 1 is selected from the group consisting of:
(±)-[3-Methyl-4-(pyridin-3-yloxy)-phenyl]-(6-piperidin-3-ylethynyl-quinazolin-4-yl)-amine;
2-Methoxy-N-(3-{4-[3-methyl-4-(pyridin-3-yloxy)-phenylamino]-quinazolin-6-yl}-prop-2-ynyl)-acetamide
(±)-[3-Methyl-4-(6-methyl-pyridin-3-yloxy)-phenyl]-(6-piperidin-3-ylethynyl-quinazolin-4-yl)-amine;
[3-Methyl-4-(6-methyl-pyridin-3-yloxy)-phenyl]-(6-piperidin-4-ylethynyl-quinazolin-4-yl)-amine;
2-Methoxy-N-(3-{4-[3-methyl-4-(6-methyl-pyridin-3-yloxy)-phenylamino]-quinazolin-6-yl}-prop-2-ynyl)-acetamide;
2-Fluoro-N-(3-{4-[3-methyl-4-(6-methyl-pyridin-3-yloxy)-phenylamino]-quinazolin-6-yl}-prop-2-ynyl)-acetamide;
E-2-Methoxy-N-(3-{4-[3-methyl-4-(6-methyl-pyridin-3-yloxy)-phenylamino]-quinazolin-6-yl}-allyl)-acetamide;
[3-Methyl-4-(pyridin-3-yloxy)-phenyl]-(6-piperidin-4-ylethynyl-quinazolin-4-yl)-amine;
2-Methoxy-N-(1-{4-[3-methyl-4-(6-methyl-pyridin-3-yloxy)-phenylamino]-quinazolin-6-ylethynyl}-cyclopropyl)-acetamide;
E-N-(3-{4-[3-Chloro-4-(6-methyl-pyridin-3-yloxy)-phenylamino]-quinazolin-6-yl}-allyl)-2-methoxy-acetamide;
N-(3-{4-[3-Chloro-4-(6-methyl-pyridin-3-yloxy)-phenylamino]-quinazolin-6-yl}-prop-2-ynyl)-acetamide;
N-(3-{4-[3-Methyl-4-(6-methyl-pyridin-3-yloxy)-phenylamino]-quinazolin-6-yl}-prop-2-ynyl)-acetamide;
E-N-(3-{4-[3-Chloro-4-(6-methyl-pyridin-3-yloxy)-phenylamino]-quinazolin-6-yl}-allyl)-acetamide;
E-2-Ethoxy-N-(3-{4-[3-methyl-4-(6-methyl-pyridin-3-yloxy)-phenylamino]-quinazolin-6-yl}-allyl)-acetamide;
1-Ethyl-3-(3-{4-[3-methyl-4-(6-methyl-pyridin-3-yloxy)-phenylamino]-quinazolin-6-yl}-prop-2-ynyl) -urea;
Piperazine-1-carboxylic acid (3-{4-[3-methyl-4-(6-methyl-pyridin-3-yloxy)-phenylamino]-quinazolin-6-yl}-prop-2-ynyl)-amide;
(±)-2-Hydroxymethyl-pyrrolidine-1-carboxylic acid (3-{4-[3-methyl-4-(6-methyl-pyridin-3-yloxy)-phenylamino]-quinazolin-6-yl}-prop-2-ynyl)-amide;
2-Dimethylamino-N-(3-{4-[3-methyl-4-(pyridin-3-yloxy)-phenylamino]-quinazolin-6-yl}-prop-2-ynyl)-acetamide;
E-N-(3-{4-[3-Methyl-4-(6-methyl-pyridin-3-yloxy)-phenylamino]-quinazolin-6-yl}-allyl)-methanesulfonamide;
Isoxazole-5-carboxylic acid (3-{4-[3-methyl-4-(6-methyl-pyridin-3-yloxy)-phenylamino]-quinazolin-6-yl}-prop-2-ynyl)-amide;
1-(1,1-Dimethyl-3-{4-[3-methyl-4-(6-methyl-pyridin-3-yloxy)-phenylamino]-quinazolin-6-yl}-prop-2-ynyl)-3-ethyl-urea;
and the pharmaceutically acceptable salts, prodrugs and solvates of the foregoing compounds.

13. The composition according to claim 12, wherein the compound of formula 1 is E-2-Methoxy-N-(3-{4-[3-methyl-4-(6-methyl-pyridin-3-yloxy)-phenylamino]-quinazolin-6-yl}-allyl)-acetamide.
